SEQUENCE LISTING (140 sequences)

SEQ ID NO: 1 - protein, 5 aa, synthetic construct
  REGION 1..5: note = synthetic peptide
SSAMG

SEQ ID NO: 2 - protein, 17 aa, synthetic construct
  REGION 1..17: note = synthetic peptide
TIVNSITTTYYADSVKG

SEQ ID NO: 3 - protein, 8 aa, synthetic construct
  REGION 1..8: note = synthetic peptide
LAGPYYDY

SEQ ID NO: 4 - protein, 5 aa, synthetic construct
  REGION 1..5: note = synthetic peptide
DYAMS

SEQ ID NO: 5 - protein, 17 aa, synthetic construct
  REGION 1..17: note = synthetic peptide
AISWNGGSTYHAESMKG

SEQ ID NO: 6 - protein, 12 aa, synthetic construct
  REGION 1..12: note = synthetic peptide
NLYGSSWYEYDY

SEQ ID NO: 7 - protein, 7 aa, synthetic construct
  REGION 1..7: note = synthetic peptide
TGYYAWS

SEQ ID NO: 8 - protein, 16 aa, synthetic construct
  REGION 1..16: note = synthetic peptide
FIARDGSTSYSPSLKS

SEQ ID NO: 9 - protein, 17 aa, synthetic construct
  REGION 1..17: note = synthetic peptide
AGEGRSWYPGYYYGMDY

SEQ ID NO: 10 - protein, 7 aa, synthetic construct
  REGION 1..7: note = synthetic peptide
TGYYAWS

SEQ ID NO: 11 - protein, 16 aa, synthetic construct
  REGION 1..16: note = synthetic peptide
VVYSDGSTSYSPSLES

SEQ ID NO: 12 - protein, 17 aa, synthetic construct
  REGION 1..17: note = synthetic peptide
DRAEARLRVGPTGGNDY

SEQ ID NO: 13 - protein, 7 aa, synthetic construct
  REGION 1..7: note = synthetic peptide
TSYYGWG

SEQ ID NO: 14 - protein, 16 aa, synthetic construct
  REGION 1..16: note = synthetic peptide
VIFSDGGTTYSPSLKS

SEQ ID NO: 15 - protein, 17 aa, synthetic construct
  REGION 1..17: note = synthetic peptide
VQYYSGSYSYPTHIYDY

SEQ ID NO: 16 - protein, 7 aa, synthetic construct
  REGION 1..7: note = synthetic peptide
TRYYAWS

SEQ ID NO: 17 - protein, 16 aa, synthetic construct
  REGION 1..16: note = synthetic peptide
LIDYDGSVYYSPSLKS

SEQ ID NO: 18 - protein, 10 aa, synthetic construct
  REGION 1..10: note = synthetic peptide
VSAATLFLDY

SEQ ID NO: 19 - protein, 7 aa, synthetic construct
  REGION 1..7: note = synthetic peptide
TSSYGWM

SEQ ID NO: 20 - protein, 16 aa, synthetic construct
  REGION 1..16: note = synthetic peptide
VIFSDGSTAYNPSLRS

SEQ ID NO: 21 - protein, 17 aa, synthetic construct
  REGION 1..17: note = synthetic peptide
SFVRGVVIGELDYGMDY

SEQ ID NO: 22 - protein, 11 aa, synthetic construct
  REGION 1..11: note = synthetic peptide
QGGSLGSYDAH

SEQ ID NO: 23 - protein, 7 aa, synthetic construct
  REGION 1..7: note = synthetic peptide
DNNSRPS

SEQ ID NO: 24 - protein, 11 aa, synthetic construct
  REGION 1..11: note = synthetic peptide
QSYDSTSDALV

SEQ ID NO: 25 - protein, 11 aa, synthetic construct
  REGION 1..11: note = synthetic peptide
QGDILESYGAS

SEQ ID NO: 26 - protein, 7 aa, synthetic construct
  REGION 1..7: note = synthetic peptide
GDDSRPS

SEQ ID NO: 27 - protein, 11 aa, synthetic construct
  REGION 1..11: note = synthetic peptide
LSADSSDYNAV

SEQ ID NO: 28 - protein, 14 aa, synthetic construct
  REGION 1..14: note = synthetic peptide
GLSSGSVTSSNYPA

SEQ ID NO: 29 - protein, 7 aa, synthetic construct
  REGION 1..7: note = synthetic peptide
NTNSRHS

SEQ ID NO: 30 - protein, 9 aa, synthetic construct
  REGION 1..9: note = synthetic peptide
HLHEGSTGV

SEQ ID NO: 31 - protein, 14 aa, synthetic construct
  REGION 1..14: note = synthetic peptide
GLSSGSVTSSNYPG

SEQ ID NO: 32 - protein, 9 aa, synthetic construct
  REGION 1..9: note = synthetic peptide
HLHKGSTGV

SEQ ID NO: 33 - protein, 14 aa, synthetic construct
  REGION 1..14: note = synthetic peptide
GLSSESVTSSNYPD

SEQ ID NO: 34 - protein, 7 aa, synthetic construct
  REGION 1..7: note = synthetic peptide
TTNSRHS

SEQ ID NO: 35 - protein, 9 aa, synthetic construct
  REGION 1..9: note = synthetic peptide
GLYMGSTAV

SEQ ID NO: 36 - protein, 14 aa, synthetic construct
  REGION 1..14: note = synthetic peptide
GLSSGSVTTSNYPG

SEQ ID NO: 37 - protein, 7 aa, synthetic construct
  REGION 1..7: note = synthetic peptide
STSSRHS

SEQ ID NO: 38 - protein, 9 aa, synthetic construct
  REGION 1..9: note = synthetic peptide
ALHMGSSTV

SEQ ID NO: 39 - protein, 11 aa, synthetic construct
  REGION 1..11: note = synthetic peptide
QGGSLGSSYAH

SEQ ID NO: 40 - protein, 7 aa, synthetic construct
  REGION 1..7: note = synthetic peptide
DDDSRPS

SEQ ID NO: 41 - protein, 10 aa, synthetic construct
  REGION 1..10: note = synthetic peptide
QSYDSSANFV

SEQ ID NO: 42 - protein, 127 aa, synthetic construct
  REGION 1..127: note = synthetic peptide
EVQLVESGPGLVKPSQTLSLTCTVSGGSITTGYYAWSWIRQPPGKGLEWMGFIARDGSTSYSPSLKSRTSISRDTSKNQFSLQLSSVTPEDTAVYYCARAGEGRSWYPGYYYGMDYWGKGTLVTVSS

SEQ ID NO: 43 - protein, 127 aa, synthetic construct
  REGION 1..127: note = synthetic peptide
EVQLQESGPGLVKPSQTVSLTCTVSGGSITTRYYGWSWIRQPPGKGLEWMGVVYSDGSTSYSPSLESRTSISRDTSKNQFSLQLSFVTPEDTAVYYCARDRAEARLRVGPTGGNDYWGQGTQVTVSS

SEQ ID NO: 44 - protein, 117 aa, synthetic construct
  REGION 1..117: note = synthetic peptide
QVQLVESGGGLVQPGGSLRLSCAASGFTFSSSAMGWVRQAPGKGLEWVSTIVNSITTTYYADSVKGRFTISTDNAKNTLYLQIDSLKSEDTAVYYCGSLAGPYYDYWGQGTQVTVSS

SEQ ID NO: 45 - protein, 121 aa, synthetic construct
  REGION 1..121: note = synthetic peptide
QVQLVESGGGLVQPGGSLRLSCAASGFTFDDYAMSWVRQAPGKGLEWVSAISWNGGSTYHAESMKGRFTISRDNAKNTLYLQMNSLKSEDTAVYYCAKNLYGSSWYEYDYWGQGTQVTVSS

SEQ ID NO: 46 - protein, 127 aa, synthetic construct
  REGION 1..127: note = synthetic peptide
EVQLVESGPGLVKPSQTLSLTCTVSGDSITTSYYGWGWIRQPPGKGLEWMGVIFSDGGTTYSPSLKSRTSISRDTSKSQFSLQLSSVTPEDTAVYYCARVQYYSGSYSYPTHIYDYWSQGTQVTVSS

SEQ ID NO: 47 - protein, 120 aa, synthetic construct
  REGION 1..120: note = synthetic peptide
QVQVQESGPGLVKPSQTLSLTCTVSGGPITTRYYAWSWIRQPPGKGLEWMGLIDYDGSVYYSPSLKSRTSISRDTAKNQFSLQLSSVTPEDTAVYYCARVSAATLFLDYWGQGTQVTVSS

SEQ ID NO: 48 - protein, 127 aa, synthetic construct
  REGION 1..127: note = synthetic peptide
EVQVQESGPGLVKPSQTLSLTCTVSGGSITTSSYGWMWIRQPPGKGLEWMGVIFSDGSTAYNPSLRSRTSISRDTSKNQFSLQLSSVTPEDTAVYYCARSFVRGVVIGELDYGMDYWGKGTLVTVSS

SEQ ID NO: 49 - protein, 127 aa, synthetic construct
  REGION 1..127: note = synthetic peptide
QLQLVESGPGLVKPSQTLSLTCTVSGGSITTGYYAWSWIRQPPGKGLEWMGFIARDGSTSYSPSLKSRTSISRDTSKNQFSLQLSSVTPEDTAVYYCARAGEGRSWYPGYYYGMDYWGKGTLVTVSS

SEQ ID NO: 50 - protein, 110 aa, synthetic construct
  REGION 1..110: note = synthetic peptide
AQAVVTQEPSLSVSPGGTVTLTCGLSSGSVTTSNYPGWFQQTPGQAPRTLIYSTSSRHSGVPSRFSGSMSGNKAALTITGAQPEDEADYYCALHMGSSTVFGGGTHLTVL

SEQ ID NO: 51 - protein, 110 aa, synthetic construct
  REGION 1..110: note = synthetic peptide
AQAVVTQEPSLSVSPGGTVTLTCGLSSGSVTSSNYPAWYQQTPGQAPRVLIYNTNSRHSGVPSRYSGFISGNKAALTITGAEPEDEADYYCHLHEGSTGVFGGGTHLNVL

SEQ ID NO: 52 - protein, 109 aa, synthetic construct
  REGION 1..109: note = synthetic peptide
ALSYELTQPSALSVTLGQTAKITCQGGSLGSSYAHWYQQKPDQAPILVIYDDDSRPSGIPERFSGSSSGGTATLTISGAQAEDEGDYYCQSYDSSANFVFGGGTKLTVL

SEQ ID NO: 53 - protein, 110 aa, synthetic construct
  REGION 1..110: note = synthetic peptide
ALSYELTQPSAVSTSLGQTARVTCQGDILESYGASWYQQKPGQAPVLVIYGDDSRPSGIPERFSGSSSGGTATLTISGAQAEDEADYYCLSADSSDYNAVFGGGTHLTVL

SEQ ID NO: 54 - protein, 110 aa, synthetic construct
  REGION 1..110: note = synthetic peptide
ALSSALTQPSTVSVSLGQTARITCQGGSLGSYDAHWYQQKPGQAPVLVIHDNNSRPSGIPERFTGSRSGETATLTISGAQTEDEADYYCQSYDSTSDALVFGGGTQLTVL

SEQ ID NO: 55 - protein, 110 aa, synthetic construct
  REGION 1..110: note = synthetic peptide
AQAVVTQEPSLSVSPGGTVTLTCGLSSESVTSSNYPDWYQQTPGQVPRLLIYTTNSRHSGVPSRFSGSISGNKAALTITGAQPEDEADYYCGLYMGSTAVFGGGTKLTVL

SEQ ID NO: 56 - protein, 110 aa, synthetic construct
  REGION 1..110: note = synthetic peptide
AQAVVTQEPSLSVSPGGTVTLTCGLSSGSVTSSNYPGWYQQTPGQAPRVLIYNTNSRHSGVPSRYSGFISGNKAALTITGAEPEDEADYYCHLHKGSTGVFGGGTHLTVL

SEQ ID NO: 57 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 58 - protein, 30 aa, synthetic construct
  REGION 1..30: note = synthetic peptide
QVQLVESGGGLVQPGGSLRLSCAASGFTFS

SEQ ID NO: 59 - protein, 14 aa, synthetic construct
  REGION 1..14: note = synthetic peptide
WVRQAPGKGLEWVS

SEQ ID NO: 60 - protein, 32 aa, synthetic construct
  REGION 1..32: note = synthetic peptide
RFTISTDNAKNTLYLQIDSLKSEDTAVYYCGS

SEQ ID NO: 61 - protein, 10 aa, synthetic construct
  REGION 1..10: note = synthetic peptide
WGQGTQVTVS

SEQ ID NO: 62 - protein, 30 aa, synthetic construct
  REGION 1..30: note = synthetic peptide
QVQLVESGGGLVQPGGSLRLSCAASGFTFD

SEQ ID NO: 63 - protein, 14 aa, synthetic construct
  REGION 1..14: note = synthetic peptide
WVRQAPGKGLEWVS

SEQ ID NO: 64 - protein, 32 aa, synthetic construct
  REGION 1..32: note = synthetic peptide
RFTISRDNAKNTLYLQMNSLKSEDTAVYYCAK

SEQ ID NO: 65 - protein, 10 aa, synthetic construct
  REGION 1..10: note = synthetic peptide
WGQGTQVTVS

SEQ ID NO: 66 - protein, 30 aa, synthetic construct
  REGION 1..30: note = synthetic peptide
EVQLVESGPGLVKPSQTLSLTCTVSGGSIT

SEQ ID NO: 67 - protein, 14 aa, synthetic construct
  REGION 1..14: note = synthetic peptide
WIRQPPGKGLEWMG

SEQ ID NO: 68 - protein, 32 aa, synthetic construct
  REGION 1..32: note = synthetic peptide
RTSISRDTSKNQFSLQLSSVTPEDTAVYYCAR

SEQ ID NO: 69 - protein, 10 aa, synthetic construct
  REGION 1..10: note = synthetic peptide
WGKGTLVTVS

SEQ ID NO: 70 - protein, 30 aa, synthetic construct
  REGION 1..30: note = synthetic peptide
EVQLQESGPGLVKPSQTVSLTCTVSGGSIT

SEQ ID NO: 71 - protein, 14 aa, synthetic construct
  REGION 1..14: note = synthetic peptide
WIRQPPGKGLEWMG

SEQ ID NO: 72 - protein, 32 aa, synthetic construct
  REGION 1..32: note = synthetic peptide
RTSISRDTSKNQFSLQLSFVTPEDTAVYYCAR

SEQ ID NO: 73 - protein, 10 aa, synthetic construct
  REGION 1..10: note = synthetic peptide
WGQGTQVTVS

SEQ ID NO: 74 - protein, 30 aa, synthetic construct
  REGION 1..30: note = synthetic peptide
EVQLVESGPGLVKPSQTLSLTCTVSGDSIT

SEQ ID NO: 75 - protein, 14 aa, synthetic construct
  REGION 1..14: note = synthetic peptide
WIRQPPGKGLEWMG

SEQ ID NO: 76 - protein, 32 aa, synthetic construct
  REGION 1..32: note = synthetic peptide
RTSISRDTSKSQFSLQLSSVTPEDTAVYYCAR

SEQ ID NO: 77 - protein, 10 aa, synthetic construct
  REGION 1..10: note = synthetic peptide
WSQGTQVTVS

SEQ ID NO: 78 - protein, 30 aa, synthetic construct
  REGION 1..30: note = synthetic peptide
QVQVQESGPGLVKPSQTLSLTCTVSGGPIT

SEQ ID NO: 79 - protein, 14 aa, synthetic construct
  REGION 1..14: note = synthetic peptide
WIRQPPGKGLEWMG

SEQ ID NO: 80 - protein, 32 aa, synthetic construct
  REGION 1..32: note = synthetic peptide
RTSISRDTAKNQFSLQLSSVTPEDTAVYYCAR

SEQ ID NO: 81 - protein, 10 aa, synthetic construct
  REGION 1..10: note = synthetic peptide
WGQGTQVTVS

SEQ ID NO: 82 - protein, 30 aa, synthetic construct
  REGION 1..30: note = synthetic peptide
EVQVQESGPGLVKPSQTLSLTCTVSGGSIT

SEQ ID NO: 83 - protein, 14 aa, synthetic construct
  REGION 1..14: note = synthetic peptide
WIRQPPGKGLEWMG

SEQ ID NO: 84 - protein, 32 aa, synthetic construct; identical to SEQ ID NO: 68
  REGION 1..32: note = synthetic peptide

SEQ ID NO: 85 - protein, 10 aa, synthetic construct
  REGION 1..10: note = synthetic peptide
WGKGTLVTVS

SEQ ID NO: 86 - protein, 30 aa, synthetic construct
  REGION 1..30: note = synthetic peptide
QLQLVESGPGLVKPSQTLSLTCTVSGGSIT

SEQ ID NO: 87 - protein, 14 aa, synthetic construct
  REGION 1..14: note = synthetic peptide
WIRQPPGKGLEWMG

SEQ ID NO: 88 - protein, 32 aa, synthetic construct; identical to SEQ ID NO: 68
  REGION 1..32: note = synthetic peptide

SEQ ID NO: 89 - protein, 10 aa, synthetic construct
  REGION 1..10: note = synthetic peptide
WGKGTLVTVS

SEQ ID NO: 90 - protein, 22 aa, synthetic construct
  REGION 1..22: note = synthetic peptide
SYELTQPSALSVTLGQTAKITC

SEQ ID NO: 91 - protein, 15 aa, synthetic construct
  REGION 1..15: note = synthetic peptide
WYQQKPDQAPILVIY

SEQ ID NO: 92 - protein, 32 aa, synthetic construct
  REGION 1..32: note = synthetic peptide
GIPERFSGSSSGGTATLTISGAQAEDEGDYYC

SEQ ID NO: 93 - protein, 10 aa, synthetic construct
  REGION 1..10: note = synthetic peptide
FGGGTKLTVL

SEQ ID NO: 94 - protein, 22 aa, synthetic construct
  REGION 1..22: note = synthetic peptide
SYELTQPSAVSTSLGQTARVTC

SEQ ID NO: 95 - protein, 15 aa, synthetic construct
  REGION 1..15: note = synthetic peptide
WYQQKPGQAPVLVIY

SEQ ID NO: 96 - protein, 32 aa, synthetic construct
  REGION 1..32: note = synthetic peptide
GIPERFSGSSSGGTATLTISGAQAEDEADYYC

SEQ ID NO: 97 - protein, 10 aa, synthetic construct
  REGION 1..10: note = synthetic peptide
FGGGTHLTVL

SEQ ID NO: 98 - protein, 22 aa, synthetic construct
  REGION 1..22: note = synthetic peptide
SSALTQPSTVSVSLGQTARITC

SEQ ID NO: 99 - protein, 15 aa, synthetic construct
  REGION 1..15: note = synthetic peptide
WYQQKPGQAPVLVIH

SEQ ID NO: 100 - protein, 32 aa, synthetic construct
  REGION 1..32: note = synthetic peptide
GIPERFTGSRSGETATLTISGAQTEDEADYYC

SEQ ID NO: 101 - protein, 10 aa, synthetic construct
  REGION 1..10: note = synthetic peptide
FGGGTQLTVL

SEQ ID NO: 102 - protein, 22 aa, synthetic construct
  REGION 1..22: note = synthetic peptide
QAVVTQEPSLSVSPGGTVTLTC

SEQ ID NO: 103 - protein, 15 aa, synthetic construct
  REGION 1..15: note = synthetic peptide
WFQQTPGQAPRTLIY

SEQ ID NO: 104 - protein, 32 aa, synthetic construct
  REGION 1..32: note = synthetic peptide
GVPSRFSGSMSGNKAALTITGAQPEDEADYYC

SEQ ID NO: 105 - protein, 10 aa, synthetic construct
  REGION 1..10: note = synthetic peptide
FGGGTHLTVL

SEQ ID NO: 106 - protein, 22 aa, synthetic construct
  REGION 1..22: note = synthetic peptide
QAVVTQEPSLSVSPGGTVTLTC

SEQ ID NO: 107 - protein, 15 aa, synthetic construct
  REGION 1..15: note = synthetic peptide
WYQQTPGQAPRVLIY

SEQ ID NO: 108 - protein, 32 aa, synthetic construct
  REGION 1..32: note = synthetic peptide
GVPSRYSGFISGNKAALTITGAEPEDEADYYC

SEQ ID NO: 109 - protein, 10 aa, synthetic construct
  REGION 1..10: note = synthetic peptide
FGGGTHLNVL

SEQ ID NO: 110 - protein, 22 aa, synthetic construct
  REGION 1..22: note = synthetic peptide
QAVVTQEPSLSVSPGGTVTLTC

SEQ ID NO: 111 - protein, 15 aa, synthetic construct
  REGION 1..15: note = synthetic peptide
WYQQTPGQVPRLLIY

SEQ ID NO: 112 - protein, 32 aa, synthetic construct
  REGION 1..32: note = synthetic peptide
GVPSRFSGSISGNKAALTITGAQPEDEADYYC

SEQ ID NO: 113 - protein, 10 aa, synthetic construct
  REGION 1..10: note = synthetic peptide
FGGGTKLTVL

SEQ ID NO: 114 - protein, 22 aa, synthetic construct
  REGION 1..22: note = synthetic peptide
QAVVTQEPSLSVSPGGTVTLTC

SEQ ID NO: 115 - protein, 15 aa, synthetic construct
  REGION 1..15: note = synthetic peptide
WFQQTPGQAPRTLIY

SEQ ID NO: 116 - protein, 32 aa, synthetic construct; identical to SEQ ID NO: 104
  REGION 1..32: note = synthetic peptide

SEQ ID NO: 117 - protein, 10 aa, synthetic construct
  REGION 1..10: note = synthetic peptide
FGGGTHLTVL

SEQ ID NO: 118 - protein, 22 aa, synthetic construct
  REGION 1..22: note = synthetic peptide
QAVVTQEPSLSVSPGGTVTLTC

SEQ ID NO: 119 - protein, 15 aa, synthetic construct
  REGION 1..15: note = synthetic peptide
WYQQTPGQAPRVLIY

SEQ ID NO: 120 - protein, 32 aa, synthetic construct; identical to SEQ ID NO: 108
  REGION 1..32: note = synthetic peptide

SEQ ID NO: 121 - protein, 10 aa, synthetic construct
  REGION 1..10: note = synthetic peptide
FGGGTHLTVL

SEQ ID NO: 122 - protein, 7 aa, synthetic construct
  REGION 1..7: note = synthetic peptide
TSYYDWS

SEQ ID NO: 123 - protein, 16 aa, synthetic construct
  REGION 1..16: note = synthetic peptide
AIAYSGNAYYSPSLKS

SEQ ID NO: 124 - protein, 13 aa, synthetic construct
  REGION 1..13: note = synthetic peptide
EDHYSDTHGWNDY

SEQ ID NO: 125 - protein, 11 aa, synthetic construct
  REGION 1..11: note = synthetic peptide
QGGSIGNFGAT

SEQ ID NO: 126 - protein, 7 aa, synthetic construct
  REGION 1..7: note = synthetic peptide
GEHSRPS

SEQ ID NO: 127 - protein, 11 aa, synthetic construct
  REGION 1..11: note = synthetic peptide
QSFDYIGNDHV

SEQ ID NO: 128 - protein, 123 aa, synthetic construct
  REGION 1..123: note = synthetic peptide
QVQLVESGPGLVQPSQTLSLTCTVSGGSITTSYYDWSWIRQPPGKGLEWMGAIAYSGNAYYSPSLKSRTSISRDTSKNQFTLQLSSVTPEDTAVYYCAREDHYSDTHGWNDYWGQGTQVTVSS

SEQ ID NO: 129 - protein, 108 aa, synthetic construct
  REGION 1..108: note = synthetic peptide
SSALTQPSAVSVSLGQTARITCQGGSIGNFGATWYQQKPGQAPVLLSLGEHSRPSGIPERFSGSKSGGTATLTISGAQAEDEADYYCQSFDYIGNDHVFGGGTHLTVL

SEQ ID NO: 130 - protein, 30 aa, synthetic construct
  REGION 1..30: note = synthetic peptide
QVQLVESGPGLVQPSQTLSLTCTVSGGSIT

SEQ ID NO: 131 - protein, 14 aa, synthetic construct
  REGION 1..14: note = synthetic peptide
WIRQPPGKGLEWMG

SEQ ID NO: 132 - protein, 32 aa, synthetic construct
  REGION 1..32: note = synthetic peptide
RTSISRDTSKNQFTLQLSSVTPEDTAVYYCAR

SEQ ID NO: 133 - protein, 11 aa, synthetic construct
  REGION 1..11: note = synthetic peptide
WGQGTQVTVSS

SEQ ID NO: 134 - protein, 22 aa, synthetic construct
  REGION 1..22: note = synthetic peptide
SSALTQPSAVSVSLGQTARITC

SEQ ID NO: 135 - protein, 15 aa, synthetic construct
  REGION 1..15: note = synthetic peptide
WYQQKPGQAPVLLSL

SEQ ID NO: 136 - protein, 32 aa, synthetic construct
  REGION 1..32: note = synthetic peptide
GIPERFSGSKSGGTATLTISGAQAEDEADYYC

SEQ ID NO: 137 - protein, 10 aa, synthetic construct
  REGION 1..10: note = synthetic peptide
FGGGTHLTVL

SEQ ID NO: 138 - protein, 144 aa, Mus musculus
MLVTYILASVLLFSSVLGQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 139 - protein, 19 aa, Homo sapiens
RYPLIFSRVKKSVEVLKNN

SEQ ID NO: 140 - protein, 19 aa, Mus musculus
RLLPVFHRVKRIVEVLKNI

(19) United States
(12) Patent Application Publication
(10) Pub. No.: US 2024/0109960 A1
(43) Pub. Date: Apr. 4, 2024

(54) ANTI-IL-9 ANTIBODIES AND METHODS OF USE THEREOF

(71) Applicant: ARGENX BV, Ghent (BE)

(72) Inventors: Christophe BLANCHETOT, Ghent (BE); Marie GODAR, Ghent (BE)

(21) Appl. No.: 18/539,678

(22) Filed: Dec. 14, 2023

Related U.S. Application Data
(63) Continuation of application No. PCT/EP2022/065989, filed on Jun. 13, 2022.
(60) Provisional application No. 63/202,494, filed on Jun. 14, 2021.

(51) Int. Cl. C07K 16/24; A61P 29/00

(52) U.S. Cl. CPC C07K 16/244; A61P 29/00; A61K 2039/505; C07K 2317/35; C07K 2317/55; C07K 2317/72; C07K 2317/76

(57) ABSTRACT

The instant disclosure provides isolated antibodies that specifically bind to IL-9 (e.g., human IL-9 or mouse IL-9). Also provided are pharmaceutical compositions comprising these antibodies, nucleic acids encoding these antibodies, expression vectors and host cells for making these antibodies, and methods of treating a subject using these antibodies.

### RELATED APPLICATIONS

This application is a continuation of International Application No. PCT/EP2022/065989, filed Jun. 13, 2022, which claims the benefit of U.S. Application No. 63/202,494, filed Jun. 14, 2021, each of which is incorporated by reference herein in its entirety.

### SEQUENCE LISTING

The instant application contains a sequence listing which has been submitted electronically in XML ST.26 format and is hereby incorporated by reference in its entirety. Said XML copy, created on November 20, 2023, is named 206006_SL.xml and is 176,315 bytes in size.

### FIELD

The instant disclosure relates to anti-IL-9 antibodies and methods of using the same.

### BACKGROUND

The protein interleukin-9 (IL-9) is a cytokine that is secreted by several different immune cells including Th9 cells, innate lymphoid cells 2 (ILC2), Th17 cells, mast cells, osteoblasts, NKT cells, and memory B cells. The binding of IL-9 to its receptors, including IL-9Rα activates the associated Janus Kinases (JAK)1 and JAK3, resulting in the activation of signal transducer and activator of transcription (STAT)1, STAT3, or STAT5 pathways, the mitogen-activated protein (MAP) kinase pathway, and insulin-related substrate (IRS) pathway.

This wide range of cellular sources points to a complex system of IL-9 expression and suggests the involvement of IL-9 in multiple physiological conditions and diseases. Increased IL-9 signaling has been found to play a role in several inflammatory and autoimmune diseases, including asthma, rheumatoid arthritis, multiple sclerosis, myasthenia gravis, and inflammatory bowel disease. Further, high levels of IL-9 signaling have also been found to promote the survival and proliferation of cancer cells, including melanoma and hematological cancers such as lymphoma and Hodgkin's disease.

Accordingly, therapeutic agents designed to antagonize IL-9 activity would be highly desirable.

### SUMMARY

The instant disclosure provides antibodies that specifically bind to IL-9 (e.g., human IL-9) and antagonize IL-9 activity. Also provided are pharmaceutical compositions comprising these antibodies, nucleic acids encoding these antibodies, expression vectors and host cells for making these antibodies, and methods of treating a subject using these antibodies.

An isolated antibody that specifically binds to human IL-9, the antibody comprising a heavy chain variable region comprising complementarity determining regions CDRH1, CDRH2, and CDRH3 and a light chain variable region comprising complementarity determining regions CDRL1, CDRL2, and CDRL3, wherein: CDRH1 comprises an amino acid sequence set forth in SEQ ID NO: 1, 4, 7, 10, 13, 16, 19, or 122; CDRH2 comprises an amino acid sequence set forth in SEQ ID NO: 2, 5, 8, 11, 14, 17, 20, or 123; CDRH3 comprises an amino acid sequence set forth in SEQ ID NO: 3, 6, 9, 12, 15, 18, 21, or 124; CDRL1 comprises an amino acid sequence set forth in SEQ ID NO: 22, 25, 28, 31, 33, 36, 39, or 125; CDRL2 comprises an amino acid sequence set forth in SEQ ID NO: 23, 26, 29, 34, 37, 40, or 126; and CDRL3 comprises an amino acid sequence set forth in SEQ ID NO: 24, 27, 30, 32, 35, 38, 41, or 127.

In one aspect, the instant disclosure provides an isolated antibody that specifically binds to human IL-9, the antibody comprising:
(a) a VH comprising the CDRH1, CDRH2, and CDRH3 amino acid sequences of the VH amino acid sequence of SEQ ID NO: 42, 43, 44, 45, 46, 47, 48, 49, or 128; and/or
(b) a VL comprising the CDRL1, CDRL2, and CDRL3 amino acid sequences of the VL amino acid sequence of SEQ ID NO: 50, 51, 52, 53, 54, 55, 56, or 129.

In an embodiment, the CDRH1, CDRH2 and CDRH3 comprise the CDRH1, CDRH2 and CDRH3 amino acid sequences, respectively, set forth in SEQ ID NOs: 1, 2, and 3; 4, 5, and 6; 7, 8, and 9; 10, 11, and 12; 13, 14, and 15; 16, 17, and 18; 19, 20, and 21; or 122, 123, and 124.

In an embodiment, the CDRL1, CDRL2 and CDRL3 comprise the CDRL1, CDRL2 and CDRL3 amino acid sequences, respectively, set forth in SEQ ID NOs: 22, 23, and 24; 25, 26, and 27; 28, 29, and 30; 31, 29, and 32; 33, 34, and 35; 36, 37, and 38; 39, 40, and 41; or 125, 126, and 127.

In an embodiment, the antibody comprises the CDRH1, CDRH2, CDRH3, CDRL1, CDRL2, and CDRL3 amino acid sequences, respectively, set forth in SEQ ID NOs: 1, 2, 3, 22, 23, and 24; 4, 5, 6, 25, 26, and 27; 7, 8, 9, 28, 29, and 30; 7, 8, 9, 31, 29, and 32; 10, 11, 12, 33, 34, and 35; 13, 14, 15, 36, 37, and 38; 16, 17, 18, 39, 40, and 41; 19, 20, 21, 36, 37, and 38; or 122, 123, 124, 125, 126, and 127.

In an embodiment, the antibody comprises the VH amino acid sequence of SEQ ID NO: 42, 43, 44, 45, 46, 47, 48, 49, or 128. In an embodiment, the amino acid sequence of the VH consists of the amino acid sequence of SEQ ID NO: 42, 43, 44, 45, 46, 47, 48, 49, or 128.

In an embodiment, the antibody comprises the VL amino acid sequence of SEQ ID NO: 50, 51, 52, 53, 54, 55, 56, or 129. In an embodiment, the amino acid sequence of the VL consists of the amino acid sequence of SEQ ID NO: 50, 51, 52, 53, 54, 55, 56, or 129.

In one aspect, the instant disclosure provides an isolated antibody that specifically binds to human IL-9, the antibody comprising a VH comprising the amino acid sequence of SEQ ID NO: 42, 43, 44, 45, 46, 47, 48, 49, or 128, and a VL comprising the amino acid sequences of SEQ ID NO 50, 51, 52, 53, 54, 55, 56, or 129.

In an embodiment, the amino acid sequence of the VH consists of the amino acid sequence of SEQ ID NO: 42, 43, 44, 45, 46, 47, 48, 49, or 128, and the amino acid sequence of the VL consists of the amino acid sequence of SEQ ID NO: 50, 51, 52, 53, 54, 55, 56, or 129.

In an embodiment, the antibody comprises the VH and VL amino acid sequences of SEQ ID NOs: 42 and 51; 43 and 55; 44 and 54; 45 and 53; 46 and 50; 47 and 52; 48 and 50; 49 and 56; or 128 and 129, respectively. In an embodiment, the amino acid sequences of the VH and VL consists of the amino acid sequence of SEQ ID NOs: 42 and 51; 43 and 55; 44 and 54; 45 and 53; 46 and 50; 47 and 52; 48 and 50; 49 and 56; or 128 and 129, respectively.

In one aspect, the instant disclosure provides an isolated antibody that specifically binds to human IL-9, wherein when bound to IL-9, the antibody binds to at least residue R91 of human IL-9. In an aspect, the instant disclosure provides an isolated antibody that specifically binds to one or more of the amino acid(s) of human IL-9 selected from the group consisting of R84, Y85, P86, L87, 188, F89, S90, R91, and K94. In an aspect, the instant disclosure provides an isolated antibody that specifically binds to one or more of the amino acid(s) of human IL-9 selected from the group consisting of L87, 188, R91, K94, S95, and V98. In an aspect, the instant disclosure provides an isolated antibody that specifically binds to one or both of the amino acid(s) of human IL-9 selected from the group consisting of 188 and R91. In an aspect, the instant disclosure provides an isolated antibody that specifically binds to one or more of the amino acid(s) of mouse IL-9 selected from the group consisting of R84, P87, V88, H90, R91, R94, 195, V98, and L99.

In an embodiment, the antibody comprises a heavy chain constant region selected from the group consisting of human IgG1, IgG2, IgG3, IgG4, IgA1, and IgA2.

In an embodiment, the antibody comprises a heavy chain constant region that is a variant of a wild-type heavy chain constant region, wherein the variant heavy chain constant region binds to an FcγR with higher affinity than the wild-type heavy chain constant region binds to the FcγR. In an embodiment, the FcγR is FcγRIIB or FcγRIIIA.

In an embodiment, the amino acid at position 297 of the heavy chain constant region, according to the EU numbering system, is A or Q. In an embodiment, the amino acids at positions 234 and 235 of the heavy chain constant region, according to the EU numbering system, are both A. In an embodiment, the amino acids at positions 433, 434, and 436 of the heavy chain constant region, according to the EU numbering system, are K, F, and Y, respectively. In an embodiment, the amino acids at positions 252, 254, and 256 of the heavy chain constant region, according to the EU numbering system, are Y, T, and E, respectively. In an embodiment, the amino acids at positions 428 and 434 of the heavy chain constant region, according to the EU numbering system, are L and S, respectively. In an embodiment, the amino acid at positions 309, 311, and 434 of the heavy chain constant region, according to the EU numbering system, are D, H, and S, respectively.

In one aspect, the instant disclosure provides an isolated antibody that cross-competes for binding to human IL-9 with an antibody disclosed herein. In one aspect, the instant disclosure provides an isolated antibody that binds to the same epitope of human IL-9 as an antibody disclosed herein.

In an embodiment, the antibody inhibits binding of human IL-9 to human IL-9Ra. In an embodiment, the antibody binds to human IL-9 with a KD of less than 1 nM. In an embodiment, the antibody is bispecific. In an embodiment, the antibody is conjugated to a cytotoxic agent, cytostatic agent, toxin, radionuclide, or detectable label.

In one aspect, the instant disclosure provides an isolated polynucleotide encoding the VH and/or the VL, or a heavy chain and/or a light chain, of an isolated antibody disclosed herein.

In one aspect, the instant disclosure provides a vector comprising a polynucleotide disclosed herein.

In one aspect, the instant disclosure provides a recombinant host cell comprising:
(a) a polynucleotide disclosed herein;
(b) a vector disclosed herein;
(c) a polynucleotide encoding the VH and the VL, or a heavy chain and a light chain, of an isolated antibody disclosed herein;
(d) a vector comprising a polynucleotide encoding the VH and the VL, or a heavy chain and a light chain, of an isolated antibody disclosed herein;
(e) a first polynucleotide encoding the VH or a heavy chain of an isolated antibody disclosed herein, and a second polynucleotide encoding the VL or a light chain of an isolated antibody disclosed herein; or
(f) a first vector comprising a first polynucleotide encoding the VH or a heavy chain of an isolated antibody disclosed herein, and a second vector comprising a second polynucleotide encoding the VL or a light chain of an isolated antibody disclosed herein.

In one aspect, the instant disclosure provides a pharmaceutical composition comprising an isolated antibody disclosed herein, the polynucleotide disclosed herein, the vector disclosed herein, or the host cell disclosed herein, and a pharmaceutically acceptable carrier or excipient.

In one aspect, the instant disclosure provides a method of producing an isolated antibody, the method comprising culturing a host cell disclosed herein under suitable conditions so that the polynucleotide is expressed, and the isolated antibody is produced.

In one aspect, the instant disclosure provides a method of producing an isolated antibody, the method comprising expressing in a cell:
(a) a first polynucleotide encoding the VH of an antibody disclosed herein and a second polynucleotide encoding the VL of an antibody disclosed herein; or
(b) a first polynucleotide encoding a heavy chain of an antibody disclosed herein and a second polynucleotide encoding a light chain of an antibody disclosed herein, under suitable conditions so that the polynucleotides are expressed, and the antibody is produced.

In one aspect, the instant disclosure provides method of antagonizing the interaction of human or mouse IL-9 with an IL-9 receptor in a subject, the method comprising administering to the subject an effective amount of an isolated antibody disclosed herein, a polynucleotide disclosed herein, a vector disclosed herein, a host cell disclosed herein, or a pharmaceutical composition disclosed herein.

In one aspect, the instant disclosure provides a method of treating an inflammatory disease in a subject, the method comprising administering to the subject an effective amount of an isolated antibody disclosed herein, a polynucleotide disclosed herein, a vector disclosed herein, a host cell disclosed herein, or a pharmaceutical composition disclosed herein.

In one aspect, the instant disclosure provides method of treating cancer in a subject, the method comprising administering to the subject an effective amount of an isolated antibody disclosed herein, a polynucleotide disclosed herein, a vector disclosed herein, a host cell disclosed herein, or a pharmaceutical composition disclosed herein.

In one aspect, the instant disclosure provides a method of treating an autoimmune disease in a subject, the method comprising administering to the subject an effective amount of an isolated antibody disclosed herein, a polynucleotide disclosed herein, a vector disclosed herein, a host cell disclosed herein, or a pharmaceutical composition disclosed herein.

In an embodiment, the isolated antibody, polynucleotide, vector, host cell, or pharmaceutical composition is administered, systemically, intravenously, subcutaneously, intratumorally, or is delivered to a tumor draining lymph node. In an embodiment, the method further comprises administering an additional therapeutic agent to the subject.

### BRIEF DESCRIPTION OF THE DRAWINGS

FIGS. 1A and 1B are graphs showing the activity of anti-IL9 mAbs. FIG. 1A is a graph showing the neutralizing activity of an initial batch of anti-IL9 mAbs. FIG. 1B is a graph showing the neutralizing activity of a second batch of anti-IL9 mAbs.

FIG. 2 is a graph showing the affinity of anti-IL9 mabs through Bio-Layer Interferometry (BLI) analysis.

FIG. 3 is a schematic showing the design of in vivo experiments to test the anti-IL-9 mAbs in a mouse model of asthma.

FIGS. 4A-4F are cartoon representations of Fab:IL-9 complexes. FIG. 4A is a cartoon representation of the Fab 6D3:hIL-9 complex; FIG. 4B is a cartoon representation of the Fab 6E2:hIL-9 complex; and FIG. 4C is a cartoon representation of the Fab 7D6:hIL-9 complex. FIG. 4D is an overlay of the Fab 6D3:hIL-9 complex, FIG. 4E is an overlay of the Fab 6E2:hIL-9 complex, and FIG. 4F is an overlay of the Fab 7D6:hIL-9 complex. Each of FIGS. 4D, 4E, and 4F are overlays of the Fab:hIL-9 complexes with the hIL-9:hIL-9Ra complex based on the structural superposition of hIL-9 with a close-up front view and top view of the binding of the C-helix by the Fabs.

### DETAILED DESCRIPTION

The instant disclosure provides isolated anti-IL-9 antibodies. Also provided are pharmaceutical compositions comprising these antibodies, nucleic acids encoding these antibodies, expression vectors and host cells for making these antibodies, and methods of treating a subject using these antibodies.

### Definitions

As used herein, the term “IL-9” refers to interleukin-9 that in humans and mice is encoded by the IL9 gene. As used herein, the term “human IL-9” or “mouse IL-9” refers to an IL-9 protein encoded by a wild-type IL9 gene (e.g., GenBank™ accession numbers NM_000590.2 (human) or NM_008373.2 (mouse)). An exemplary amino acid sequence of a human IL-9 protein is provided as SEQ ID NO: 57. An exemplary sequence of a mouse IL-9 protein is provided as SEQ ID NO: 138.

TABLE 1
Exemplary IL-9 amino acid sequences.

| | SEQ | |
|---|---|---|
| | ID NO | Amino acid sequence |
| Human | 57 | MLLAMVLTSALLLCSVAGQGCPTLAGILDINFLI |
| IL-9 | | NKMQEDPASKCHCSANVTSCLCLGIPSDNCTRPC |
| | | FSERLSQMTNTTMQTRYPLIFSRVKKSVEVLKNN |
| | | KCPYFSCEQPCNQTTAGNALTFLKSLLEIFQKEK |
| | | MRGMRGKI |
| Mouse | 138 | MLVTYILASVLLFSSVLGQRCSTTWGIRDTNYLI |
| IL-9 | | ENLKDDPPSKCSCSGNVTSCLCLSVPTDDCTTPC |
| | | YREGLLQLTNATQKSRLLPVFHRVKRIVEVLKNI |
| | | TCPSFSCEKPCNQTMAGNTLSFLKSLLGTFQKTE |
| | | MQRQKSRP |

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, antibodies described herein are IgG antibodies, or a class (e.g., human IgG₁ or IgG₄) or subclass thereof. In an embodiment, the antibody is a humanized monoclonal antibody. In an embodiment, the antibody is a human monoclonal antibody.

“Multispecific antibodies” are antibodies (e.g., bispecific antibodies) that specifically bind to two or more different antigens or two or more different regions of the same antigen. Multispecific antibodies include bispecific antibodies that contain two different antigen-binding sites (exclusive of the Fc region). Multispecific antibodies can include, for example, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, heteroconjugate antibodies, linked single chain antibodies or linked-single-chain Fvs (scFv), camelized antibodies, affibodies, linked Fab fragments, F(ab′)₂ fragments, chemically-linked Fvs, and disulfide-linked Fvs (sdFv). Multispecific antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In an embodiment, multispecific antibodies described herein are IgG antibodies, or a class (e.g., human IgG₁, IgG₂, or IgG₄) or subclass thereof.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human and/or mouse IL-9). CDRH1, CDRH2 and CDRH3 denote the heavy chain CDRs, and CDRL1, CDRL2 and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a K_{A} that is at least 2 logs (e.g., factors of 10), 2.5 logs, 3 logs, 4 logs or greater than the K_{A} when the molecules bind non-specifically to another antigen.

As used herein, the term “EU numbering system” refers to the EU numbering convention for the constant regions of an antibody, as described in Edelman G. M et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a human or non-human mammal. In certain embodiments, the subject is a human.

As used herein with respect to an antibody or polynucleotide, the term “isolated” refers to an antibody or polynucleotide that is separated from one or more contaminants (e.g., polypeptides, polynucleotides, lipids or carbohydrates, etc.) which are present in a natural source of the antibody or polynucleotide. All instances of “isolated antibodies” described herein are additionally contemplated as antibodies that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polynucleotides” described herein are additionally contemplated as polynucleotides that may be, but need not be, isolated.

The determination of “percent identity” between two sequences (e.g., amino acid sequences or nucleic acid sequences) can be accomplished using a mathematical algorithm. A non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and)(BLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### Anti-IL-9 Antibodies

In one aspect, the instant disclosure provides antibodies that specifically bind to IL-9 (e.g., human IL-9 or mouse IL-9). The amino acid sequences of the CDR and VH/VL sequences of exemplary antibodies that specifically bind IL-9 are set forth in Tables 2 and 3, respectively.

TABLE 2
Amino acid sequences of the CDRs
of exemplary anti-IL-9 antibodies.

| | | SEQ | | SEQ | | SEQ |
|---|---|---|---|---|---|---|
| Anti- | | ID | | ID | | ID |
| body | | NO | | NO | | NO |
| | HCDR1 | | HCDR2 | | HCDR3 | |
| 9MP6E2 | SSAMG | 1 | TIVNSITTT | 2 | LAGPYYDY | 3 |
| | | | YYADSVKG | | | |
| 9MP6C4 | DYAMS | 4 | AISWNGGST | 5 | NLYGSSWYE | 6 |
| | | | YHAESMKG | | YDY | |
| 9MP7A4 | TGYYAWS | 7 | FIARDGSTS | 8 | AGEGRSWYP | 9 |
| | | | YSPSLKS | | GYYYGMDY | |
| 9MP7D6 | TGYYAWS | 7 | FIARDGSTS | 8 | AGEGRSWYP | 9 |
| | | | YSPSLKS | | GYYYGMDY | |
| 9MP6F2 | TRYYGWS | 10 | VVYSDGSTS | 11 | DRAEARLRV | 12 |
| | | | YSPSLES | | GPTGGNDY | |
| 9MP8G3 | TSYYGWG | 13 | VIFSDGGTT | 14 | VQYYSGSYS | 15 |
| | | | YSPSLKS | | YPTHIYDY | |
| 9MP6D3 | TRYYAWS | 16 | LIDYDGSVY | 17 | VSAATLFLD | 18 |
| | | | YSPSLKS | | Y | |
| 9MP8C3 | TSSYGWM | 19 | VIFSDGSTA | 20 | SFVRGVVIG | 21 |
| | | | YNPSLRS | | ELDYGMDY | |
| 35D8 | TSYYDWS | 122 | AIAYSGNAY | 123 | EDHYSDTHG | 124 |
| | | | YSPSLKS | | WNDY | |
| | LCDR1 | | LCDR2 | | LCDR3 | |
| 9MP6E2 | QGGSLGS | 22 | DNNSRPS | 23 | QSYDSTSDA | 24 |
| | YDAH | | | | LV | |
| 9MP6C4 | QGDILES | 25 | GDDSRPS | 26 | LSADSSDYN | 27 |
| | YGAS | | | | AV | |
| 9MP7A4 | GLSSGSV | 28 | NTNSRHS | 29 | HLHEGSTGV | 30 |
| | TSSNYPA | | | | | |
| 9MP7D6 | GLSSGSV | 31 | NTNSRHS | 29 | HLHKGSTGV | 32 |
| | TSSNYPG | | | | | |
| 9MP6F2 | GLSSESV | 33 | TTNSRHS | 34 | GLYMGSTAV | 35 |
| | TSSNYPD | | | | | |
| 9MP8G3 | GLSSGSV | 36 | STSSRHS | 37 | ALHMGSSTV | 38 |
| | TTSNYPG | | | | | |
| 9MP6D3 | QGGSLGS | 39 | DDDSRPS | 40 | QSYDSSANF | 41 |
| | SYAH | | | | V | |
| 9MP8C3 | GLSSGSV | 36 | STSSRHS | 37 | ALHMGSSTV | 38 |
| | TTSNYPG | | | | | |
| 35D8 | QGGSIGN | 125 | GEHSRPS | 126 | QSFDYIGND | 127 |
| | FGAT | | | | HV | |

TABLE 3
Amino acid sequences of the
VH/VLs of exemplary anti-IL-9 antibodies.

| | | |
|---|---|---|
| | SEQ ID | |
| Antibody | NO | sequence |

Heavy chain variable regions

| | | |
|---|---|---|
| 9MP7A4 | 42 | EVQLVESGPGLVKPSQTLSLTCTVSGGSITTGY |
| | | YAWSWIRQPPGKGLEWMGFIARDGSTSYSPSLK |
| | | SRTSISRDTSKNQFSLQLSSVTPEDTAVYYCAR |
| | | AGEGRSWYPGYYYGMDYWGKGTLVTVSS |
| 9MP6F2 | 43 | EVQLQESGPGLVKPSQTVSLTCTVSGGSITTRY |
| | | YGWSWIRQPPGKGLEWMGVVYSDGSTSYSPSLE |
| | | SRTSISRDTSKNQFSLQLSFVTPEDTAVYYCAR |
| | | DRAEARLRVGPTGGNDYWGQGTQVTVSS |
| 9MP6E2 | 44 | QVQLVESGGGLVQPGGSLRLSCAASGFTFSSSA |
| | | MGWVRQAPGKGLEWVSTIVNSITTTYYADSVKG |
| | | RFTISTDNAKNTLYLQIDSLKSEDTAVYYCGSL |
| | | AGPYYDYWGQGTQVTVSS |
| 9MP6C4 | 45 | QVQLVESGGGLVQPGGSLRLSCAASGFTFDDYA |
| | | MSWVRQAPGKGLEWVSAISWNGGSTYHAESMKG |
| | | RFTISRDNAKNTLYLQMNSLKSEDTAVYYCAKN |
| | | LYGSSWYEYDYWGQGTQVTVSS |
| 9MP8G3 | 46 | EVQLVESGPGLVKPSQTLSLTCTVSGDSITTSY |
| | | YGWGWIRQPPGKGLEWMGVIFSDGGTTYSPSLK |
| | | SRTSISRDTSKSQFSLQLSSVTPEDTAVYYCAR |
| | | VQYYSGSYSYPTHIYDYWSQGTQVTVSS |
| 9MP6D3 | 47 | QVQVQESGPGLVKPSQTLSLTCTVSGGPITTRY |
| | | YAWSWIRQPPGKGLEWMGLIDYDGSVYYSPSLK |
| | | SRTSISRDTAKNQFSLQLSSVTPEDTAVYYCAR |
| | | VSAATLFLDYWGQGTQVTVSS |
| 9MP8C3 | 48 | EVQVQESGPGLVKPSQTLSLTCTVSGGSITTSS |
| | | YGWMWIRQPPGKGLEWMGVIFSDGSTAYNPSLR |
| | | SRTSISRDTSKNQFSLQLSSVTPEDTAVYYCAR |
| | | SFVRGVVIGELDYGMDYWGKGTLVTVSS |
| 9MP7D6 | 49 | QLQLVESGPGLVKPSQTLSLTCTVSGGSITTGY |
| | | YAWSWIRQPPGKGLEWMGFIARDGSTSYSPSLK |
| | | SRTSISRDTSKNQFSLQLSSVTPEDTAVYYCAR |
| | | AGEGRSWYPGYYYGMDYWGKGTLVTVSS |
| 35D8 | 128 | QVQLVESGPGLVQPSQTLSLTCTVSGGSITTSY |
| | | YDWSWIRQPPGKGLEWMGAIAYSGNAYYSPSLK |
| | | SRTSISRDTSKNQFTLQLSSVTPEDTAVYYCAR |
| | | EDHYSDTHGWNDYWGQGTQVTVSS |

Light chain variable regions

| | | |
|---|---|---|
| 9MP8C3 | 50 | AQAVVTQEPSLSVSPGGTVTLTCGLSSGSVTTS |
| | | NYPGWFQQTPGQAPRTLIYSTSSRHSGVPSRFS |
| | | GSMSGNKAALTITGAQPEDEADYYCALHMGSST |
| | | VFGGGTHLTVL |
| 9MP7A4 | 51 | AQAVVTQEPSLSVSPGGTVTLTCGLSSGSVTSS |
| | | NYPAWYQQTPGQAPRVLIYNTNSRHSGVPSRYS |
| | | GFISGNKAALTITGAEPEDEADYYCHLHEGSTG |
| | | VFGGGTHLNVL |
| 9MP6D3 | 52 | ALSYELTQPSALSVTLGQTAKITCQGGSLGSSY |
| | | AHWYQQKPDQAPILVIYDDDSRPSGIPERFSGS |
| | | SSGGTATLTISGAQAEDEGDYYCQSYDSSANFV |
| | | FGGGTKLTVL |
| 9MP6C4 | 53 | ALSYELTQPSAVSTSLGQTARVTCQGDILESYG |
| | | ASWYQQKPGQAPVLVIYGDDSRPSGIPERFSGS |
| | | SSGGTATLTISGAQAEDEADYYCLSADSSDYNA |
| | | VFGGGTHLTVL |
| 9MP6E2 | 54 | ALSSALTQPSTVSVSLGQTARITCQGGSLGSYD |
| | | AHWYQQKPGQAPVLVIHDNNSRPSGIPERFTGS |
| | | RSGETATLTISGAQTEDEADYYCQSYDSTSDAL |
| | | VFGGGTQLTVL |
| 9MP6F2 | 55 | AQAVVTQEPSLSVSPGGTVTLTCGLSSESVTSS |
| | | NYPDWYQQTPGQVPRLLIYTTNSRHSGVPSRFS |
| | | GSISGNKAALTITGAQPEDEADYYCGLYMGSTA |
| | | VFGGGTKLTVL |
| 9MP8G3 | 50 | AQAVVTQEPSLSVSPGGTVTLTCGLSSGSVTTS |
| | | NYPGWFQQTPGQAPRTLIYSTSSRHSGVPSRFS |
| | | GSMSGNKAALTITGAQPEDEADYYCALHMGSST |
| | | VFGGGTHLTVL |
| 9MP7D6 | 56 | AQAVVTQEPSLSVSPGGTVTLTCGLSSGSVTSS |
| | | NYPGWYQQTPGQAPRVLIYNTNSRHSGVPSRYS |
| | | GFISGNKAALTITGAEPEDEADYYCHLHKGSTG |
| | | VFGGGTHLTVL |
| 35D8 | 129 | SSALTQPSAVSVSLGQTARITCQGGSIGNFGAT |
| | | WYQQKPGQAPVLLSLGEHSRPSGIPERFSGSKS |
| | | GGTATLTISGAQAEDEADYYCQSFDYIGNDHVF |
| | | GGGTHLTVL |

TABLE 4
VH and VL framework (FR) sequences
of exemplary anti-IL-9 antibodies.

| | | | | |
|---|---|---|---|---|
| | | FR2 (SEQ | | FR4 (SEQ |
| Antibody | FR1 (SEQ ID NO) | ID NO) | FR3 (SEQ ID NO) | ID NO) |

VH framework sequences

| | | | | |
|---|---|---|---|---|
| 9MP6E2 | QVQLVESGGGLVQPGGSLR | WVRQAPGKGLE | RFTISTDNAKNTLYLQIDSL | WGQGTQ |
| | LSCAASGFTFS (58) | WVS (59) | KSEDTAVYYCGS (60) | VTVS (61) |
| 9MP6C4 | QVQLVESGGGLVQPGGSLR | WVRQAPGKGLE | RFTISRDNAKNTLYLQMNSL | WGQGTQ |
| | LSCAASGFTFD (62) | WVS (63) | KSEDTAVYYCAK (64) | VTVS (65) |
| 9MP7A4 | EVQLVESGPGLVKPSQTLS | WIRQPPGKGLE | RTSISRDTSKNQFSLQLSSV | WGKGTL |
| | LTCTVSGGSIT (66) | WMG (67) | TPEDTAVYYCAR (68) | VTVS (69) |
| 9MP6F2 | EVQLQESGPGLVKPSQTVS | WIRQPPGKGLE | RTSISRDTSKNQFSLQLSFV | WGQGTQ |
| | LTCTVSGGSIT (70) | WMG (71) | TPEDTAVYYCAR (72) | VTVS (73) |
| 9MP8G3 | EVQLVESGPGLVKPSQTLS | WIRQPPGKGLE | RTSISRDTSKSQFSLQLSSV | WSQGTQ |
| | LTCTVSGDSIT (74) | WMG (75) | TPEDTAVYYCAR (76) | VTVS (77) |
| 9MP6D3 | QVQVQESGPGLVKPSQTLS | WIRQPPGKGLE | RTSISRDTAKNQFSLQLSSV | WGQGTQ |
| | LTCTVSGGPIT (78) | WMG (79) | TPEDTAVYYCAR (80) | VTVS (81) |
| 9MP8C3 | EVQVQESGPGLVKPSQTLS | WIRQPPGKGLE | RTSISRDTSKNQFSLQLSSV | WGKGTL |
| | LTCTVSGGSIT (82) | WMG (83) | TPEDTAVYYCAR (84) | VTVS (85) |
| 9MP7D6 | QLQLVESGPGLVKPSQTLS | WIRQPPGKGLE | RTSISRDTSKNQFSLQLSSV | WGKGTL |
| | LTCTVSGGSIT (86) | WMG (87) | TPEDTAVYYCAR (88) | VTVS (89) |
| 35D8 | QVQLVESGPGLVQPSQTLS | WIRQPPGKGLE | RTSISRDTSKNQFTLQLSSV | WGQGTQVT |
| | LTCTVSGGSIT (130) | WMG (131) | TPEDTAVYYCAR (132) | VSS (133) |

VL framework sequences

| | | | | |
|---|---|---|---|---|
| 9MP6D3 | SYELTQPSALSVTLGQTAK | WYQQKPDQAPI | GIPERFSGSSSGGTATLTIS | FGGGTK |
| | ITC (90) | LVIY (91) | GAQAEDEGDYYC (92) | LTVL (93) |
| 9MP6C4 | SYELTQPSAVSTSLGQTAR | WYQQKPGQAPV | GIPERFSGSSSGGTATLTIS | FGGGTH |
| | VTC (94) | LVIY (95) | GAQAEDEADYYC (96) | LTVL (97) |
| 9MP6E2 | SSALTQPSTVSVSLGQTAR | WYQQKPGQAPV | GIPERFTGSRSGETATLTIS | FGGGTQLT |
| | ITC (98) | LVIH (99) | GAQTEDEADYYC (100) | VL (101) |
| 9MF8C3 | QAVVTQEPSLSVSPGGTVT | WFQQTPGQAPR | GVPSRFSGSMSGNKAALTIT | FGGGTHLT |
| | LTC (102) | TLIY (103) | GAQPEDEADYYC (104) | VL (105) |
| 9MP7A4 | QAVVTQEPSLSVSPGGTVT | WYQQTPGQAPR | GVPSRYSGFISGNKAALTIT | FGGGTHLN |
| | LTC (106) | VLIY (107) | GAEPEDEADYYC (108) | VL (109) |
| 9MP6F2 | QAVVTQEPSLSVSPGGTVT | WYQQTPGQVPR | GVPSRFSGSISGNKAALTIT | FGGGTKLT |
| | LTC (110) | LLIY (111) | GAQPEDEADYYC (112) | VL (113) |
| 9MP8G3 | QAVVTQEPSLSVSPGGTVT | WFQQTPGQAPR | GVPSRFSGSMSGNKAALTIT | FGGGTHLT |
| | LTC (114) | TLIY (115) | GAQPEDEADYYC (116) | VL (117) |
| 9MP7D6 | QAVVTQEPSLSVSPGGTVT | WYQQTPGQAPR | GVPSRYSGFISGNKAALTIT | FGGGTHLT |
| | LTC (118) | VLIY (119) | GAEPEDEADYYC (120) | VL (121) |
| 35D8 | SSALTQPSAVSVSLGQTAR | WYQQKPGQAPV | GIPERFSGSKSGGTATLTIS | FGGGTHLT |
| | ITC (134) | LLSL (135) | GAQAEDEADYYC (136) | VL (137) |

In an embodiment, the instant disclosure provides an isolated antibody that specifically binds to IL-9 (e.g., human IL-9 or mouse IL-9), the antibody comprising a CDRH1, CDRH2, and CDRH3 set forth in Table 2. In an embodiment, the antibody comprises a CDRH1 set forth in Table 2. In an embodiment, the antibody comprises a CDRH2 set forth in Table 2. In an embodiment, the antibody comprises a CDRH3 set forth in Table 2.

In an embodiment, the instant disclosure provides an isolated antibody that specifically binds to IL-9 (e.g., human IL-9 or mouse IL-9), the antibody comprising a VH domain comprising one, two, or all three of the CDRs of a VH domain set forth in Table 3. In an embodiment, the antibody comprises the CDRH1 of a VH domain set forth in Table 3. In an embodiment, the antibody comprises the CDRH2 of a VH domain set forth in Table 3. In an embodiment, the antibody comprises the CDRH3 of a VH domain set forth in Table 3.

In an embodiment, the instant disclosure provides an isolated antibody that specifically binds to IL-9 (e.g., human IL-9 or mouse IL-9), the antibody comprising a CDRL1, CDRL2, and CDRL3 set forth in Table 2. In an embodiment, the antibody comprises a CDRL1 set forth in Table 2. In an embodiment, the antibody comprises a CDRL2 set forth in Table 2. In an embodiment, the antibody comprises a CDRL3 set forth in Table 2.

In an embodiment, the instant disclosure provides an isolated antibody that specifically binds to IL-9 (e.g., human IL-9 or mouse IL-9), the antibody comprising a VL domain comprising one, two, or all three of the CDRs of a VL domain disclosed in Table 3. In an embodiment, the antibody comprises the CDRL1 of a VL domain set forth in Table 3. In an embodiment, the antibody comprises the CDRL2 of a VL domain set forth in Table 3. In an embodiment, the antibody comprises the CDRL3 of a VL domain set forth in Table 3.

The individual CDRs of an antibody disclosed herein can be determined according to any CDR numbering scheme known in the art.

In an embodiment, one or more of the CDRs of an antibody disclosed herein can be determined according to Kabat et al., J. Biol. Chem. 252, 6609-6616 (1977) and Kabat et al., Sequences of protein of immunological interest (1991), each of which is herein incorporated by reference in its entirety.

In an embodiment, the instant disclosure provides antibodies that specifically bind to IL-9 (e.g., human IL-9 or mouse IL-9) and comprise CDRs of an antibody disclosed in Table 2 or Table 3 herein as determined by the Kabat numbering scheme.

In an embodiment, one or more of the CDRs of an antibody disclosed herein can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226, all of which are herein incorporated by reference in their entireties).

In an embodiment, the instant disclosure provides antibodies that specifically bind to IL-9 (e.g., human IL-9 or mouse IL-9) and comprise CDRs of an antibody disclosed in Table 2 or Table 3 herein, as determined by the Chothia numbering system.

In an embodiment, one or more of the CDRs of an antibody disclosed herein can be determined according to MacCallum RM et al., (1996) J Mol Biol 262: 732-745, herein incorporated by reference in its entirety. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001), herein incorporated by reference in its entirety.

In an embodiment, the instant disclosure provides antibodies that specifically bind to IL-9 (e.g., human IL-9 or mouse IL-9) and comprise CDRs of an antibody disclosed in Table 2 or Table 3 herein, as determined by the MacCallum numbering system.

In an embodiment, the CDRs of an antibody disclosed herein can be determined according to the IMGT numbering system as described in: Lefranc M-P, (1999) The Immunologist 7: 132-136; Lefranc M-P et al., (1999) Nucleic Acids Res 27: 209-212, each of which is herein incorporated by reference in its entirety; and Lefranc M-P et al., (2009) Nucleic Acids Res 37: D1006-D1012.

In an embodiment, the instant disclosure provides antibodies that specifically bind to IL-9 (e.g., human IL-9 or mouse IL-9) and comprise CDRs of an antibody disclosed in Table 2 or Table 3 herein, as determined by the IMGT numbering system.

In an embodiment, the CDRs of an antibody disclosed herein can be determined according to the AbM numbering scheme, which refers to AbM hypervariable regions, which represent a compromise between the Kabat CDRs and Chothia structural loops and are used by Oxford Molecular's AbM antibody modeling software (Oxford Molecular Group, Inc.), herein incorporated by reference in its entirety.

In an embodiment, the instant disclosure provides antibodies that specifically bind to IL-9 (e.g., human IL-9 or mouse IL-9) and comprise CDRs of an antibody disclosed in Table 2 or Table 3 herein as determined by the AbM numbering scheme.

In an embodiment, the CDRs of an antibody disclosed herein can be determined according to the AHo numbering system, as described in Honegger and Plückthun A, J. Mol. Biol. 309:657-670 (2001), herein incorporated by reference in its entirety.

In an embodiment, the instant disclosure provides antibodies that specifically bind to IL-9 (e.g., human IL-9 or mouse IL-9) and comprise CDRs of an antibody disclosed in Table 2 or Table 3 herein, as determined by the AHo numbering system.

In an embodiment, the individual CDRs of an antibody disclosed herein are each independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of IL-9.

In an embodiment, the instant disclosure provides antibodies that specifically bind to IL-9 (e.g., human IL-9 or mouse IL-9) and comprise a VH comprising the CDRH1, CDRH2, and CDRH3 region amino acid sequences of a VH set forth in SEQ ID NO: 42, 43, 44, 45, 46, 47, 48, 49, or 128, and a VL comprising the CDRL1, CDRL2, and CDRL3 region amino acid sequences of a VL set forth in SEQ ID NO: 50, 51, 52, 53, 54, 55, 56, or 129, wherein each CDR is independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of IL-9 (e.g., human IL-9 or mouse IL-9).

In an embodiment, the instant disclosure provides an isolated antibody that specifically binds to IL-9 (e.g., human IL-9 or mouse IL-9), wherein the isolated antibody comprises a VH comprising the CDRH1, CDRH2 and CDRH3 amino acid sequences, respectively, set forth in SEQ ID NOs: 1, 2, and 3; 4, 5, and 6; 7, 8, and 9; 10, 11, and 12; 13, 14, and 15; 16, 17, and 18; 19, 20, and 21; or 122, 123, and 124.

In an embodiment, the instant disclosure provides an isolated antibody that specifically binds to IL-9 (e.g., human IL-9 or mouse IL-9), wherein the isolated antibody comprises a VL comprising the CDRL1, CDRL2 and CDRL3 amino acid sequences, respectively, set forth in SEQ ID NOs: 22, 23, and 24; 25, 26, and 27; 28, 29, and 30; 31, 29, and 32; 33, 34, and 35; 36, 37, and 38; 39, 40, and 41; or 125, 126, and 127.

In an embodiment, the instant disclosure provides an isolated antibody that specifically binds to IL-9 (e.g., human IL-9 or mouse IL-9), wherein the isolated antibody comprises a VH comprising CDRH1, CDRH2, and CDRH3 regions, and a VL comprising CDRL1, CDRL2, and CDRL3 regions, wherein the CDRH1, CDRH2, CDRH3, CDRL1, CDRL2, and CDRL3 regions comprise the amino acid sequences, respectively, set forth in SEQ ID NOs: 1, 2, 3, 22, 23, and 24; 4, 5, 6, 25, 26, and 27; 7, 8, 9, 28, 29, and 30; 7, 8, 9, 31, 29, and 32; 10, 11, 12, 33, 34, and 35; 13, 14, 15, 36, 37, and 38; 16, 17, 18, 39, 40, and 41; 19, 20, 21, 36, 37, and 38; or 122, 123, 124, 125, 126, and 127.

In an embodiment, the instant disclosure provides an isolated antibody that specifically binds to IL-9 (e.g., human IL-9 or mouse IL-9) comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 42, 43, 44, 45, 46, 47, 48, 49, or 128. In an embodiment, the instant disclosure provides an isolated antibody that specifically binds to IL-9 (e.g., human IL-9 or mouse IL-9), comprising a VH comprising an amino acid sequence set forth in SEQ ID NO: 42, 43, 44, 45, 46, 47, 48, 49, or 128. In an embodiment, the amino acid sequence of the VH consists of the amino acid sequence set forth in SEQ ID NO: 42, 43, 44, 45, 46, 47, 48, 49, or 128.

In an embodiment, the instant disclosure provides an isolated antibody that specifically binds to IL-9 (e.g., human IL-9 or mouse IL-9), comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 50, 51, 52, 53, 54, 55, 56, or 129. In an embodiment, the instant disclosure provides an isolated antibody that specifically binds to IL-9 (e.g., human IL-9 or mouse IL-9), comprising a VL comprising an amino acid sequence set forth in SEQ ID NO: 50, 51, 52, 53, 54, 55, 56, or 129. In an embodiment, the amino acid sequence of the VL consists of the amino acid sequence set forth in SEQ ID NO: 50, 51, 52, 53, 54, 55, 56, or 129.

In an embodiment, the instant disclosure provides an isolated antibody that specifically binds to IL-9 (e.g., human IL-9 or mouse IL-9), comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 42, 43, 44, 45, 46, 47, 48, 49, or 128, and a VL comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 50, 51, 52, 53, 54, 55, 56, or 129. In an embodiment, the instant disclosure provides an isolated antibody that specifically binds to IL-9 (e.g., human IL-9 or mouse IL-9), comprising a VH comprising an amino acid sequence of SEQ ID NO: 42, 43, 44, 45, 46, 47, 48, 49, or 128, and a VL comprising an amino acid sequence of SEQ ID NO: 50, 51, 52, 53, 54, 55, 56, or 129. In an embodiment, the amino acid sequence of the VH consists of the amino acid sequence set forth in SEQ ID NO: 42, 43, 44, 45, 46, 47, 48, 49, or 128; and the amino acid sequence of the VL consists of the amino acid sequence set forth in SEQ ID NO: 50, 51, 52, 53, 54, 55, 56, or 129.

In an embodiment, the instant disclosure provides an isolated antibody that specifically binds to IL-9 (e.g., human IL-9 or mouse IL-9), comprising the VH and VL amino acid sequences set forth in SEQ ID NOs: 42 and 51; 43 and 55; 44 and 54; 45 and 53; 46 and 50; 47 and 52; 48 and 50; 49 and 56, or 128 and 129, respectively. In an embodiment, the amino acid sequences of VH and VL consist of the amino acid sequences set forth in SEQ ID NOs: 42 and 51; 43 and 55; 44 and 54; 45 and 53; 46 and 50; 47 and 52; 48 and 50; 49 and 56; or 128 and 129, respectively.

In an embodiment, the instant disclosure provides an isolated antibody that cross-competes for binding to IL-9 (e.g., human IL-9 or mouse IL-9) with an antibody comprising the VH and VL amino acid sequences set forth in SEQ ID NOs: 42 and 51; 43 and 55; 44 and 54; 45 and 53; 46 and 50; 47 and 52; 48 and 50; 49 and 56; or 128 and 129, respectively.

In an embodiment, the instant disclosure provides an isolated antibody that binds to the same or an overlapping epitope of IL-9 (e.g., an epitope of human IL-9 or mouse IL-9) as an antibody described herein, e.g., an antibody comprising the VH and VL amino acid sequences set forth in SEQ ID NOs: 42 and 51; 43 and 55; 44 and 54; 45 and 53; 46 and 50; 47 and 52; 48 and 50; 49 and 56; or 128 and 129, respectively.

In an embodiment, the instant disclosure provides an isolated antibody that binds to an epitope including at least residue R91 of IL-9 (e.g., an epitope of human IL-9 or mouse IL-9). In an embodiment, the isolated antibody binds one or more residues of the C-helix of human or mouse IL-9 (amino acids 84 to 102 of IL-9). The amino acid sequence of the C-helix of human IL-9 is RYPLIFSRVKKSVEVLKNN (SEQ ID NO: 139) and the amino acid sequence of the C-helix of mouse IL-9 is RLLPVFHRVKRIVEVLKNI (SEQ ID NO: 140). In an embodiment, the isolated antibody binds to one or more (e.g., 2, 3, 4, 5, 6, 7, 8, or 9) of the amino acid(s) of human IL-9 selected from the group consisting of R84, Y85, P86, L87, 188, F89, S90, R91, and K94. In an embodiment, the isolated antibody binds to one or more (e.g., 2, 3, 4, 5, or 6) of the amino acid(s) of human IL-9 selected from the group consisting of L87, 188, R91, K94, S95, and V98. In an embodiment, the isolated antibody binds to one or both of the amino acid(s) of human IL-9 selected from the group consisting of 188 and R91. In an embodiment, the isolated antibody binds to one or more (e.g., 2, 3, 4, 5, 6, 7, 8, or 9) of the amino acid(s) of mouse IL-9 selected from the group consisting of R84, P87, V88, H90, R91, R94, 195, V98, and L99. In an embodiment, the isolated antibody also binds to cynomolgous IL-9.

In an embodiment, the epitope of an antibody can be determined by, e.g., NMR spectroscopy, surface plasmon resonance (BIAcore®), X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303, all of which are herein incorporated by reference in their entireties). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.; U.S. Patent Application No. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323, all of which are herein incorporated by reference in their entireties). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In an embodiment, the epitope of an antibody is determined using alanine scanning mutagenesis studies. In addition, or antibodies that recognize and bind to the same or overlapping epitopes of IL-9 (e.g., human IL-9 or mouse IL-9) can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as IL-9 (e.g., human IL-9 or mouse IL-9). Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9: 242-253); solid phase direct biotin-avidin EIA (see Kirkland T N et al., (1986) J Immunol 137: 3614-9); solid phase direct labeled assay, solid phase direct labeled sandwich assay (see Harlow E & Lane D, (1988) Antibodies: A Laboratory Manual, Cold Spring Harbor Press); solid phase direct label RIA using 1-125 label (see Morel G A et al., (1988) Mol Immunol 25(1): 7-15); solid phase direct biotin-avidin EIA (see Cheung R C et al., (1990) Virology 176: 546-52); and direct labeled RIA (see Moldenhauer G et al., (1990) Scand J Immunol 32: 77-82), all of which are herein incorporated by reference in their entireties. Typically, such an assay involves the use of purified antigen (e.g., IL-9, such as human IL-9 or mouse IL-9) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually, the test immunoglobulin is present in excess. Usually, when a competing antibody is present in excess, it will inhibit specific binding of a reference or antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, e.g., Wagener C et al., (1983) J Immunol 130: 2308-2315; Wagener C et al., (1984) J Immunol Methods 68: 269-274; Kuroki M et al., (1990) Cancer Res 50: 4872-4879; Kuroki M et al., (1992) Immunol Invest 21: 523-538; Kuroki M et al., (1992) Hybridoma 11: 391-407 and Antibodies: A Laboratory Manual, Ed Harlow E & Lane D editors supra, pp. 386-389, all of which are herein incorporated by reference in their entireties.

In an embodiment, the antibody inhibits the binding of human IL-9 to human IL-9Ra. In an embodiment, the binding of human IL-9 to human IL-9Ra is reduced by more than 50%, 55%, 60%, 65%, 70%, 75%, 80%, 85%, 90%, 95%, 96%, 97%, 98%, or 99% in the presence of the antibody relative to the binding of human IL-9 to human IL-9Ra in the absence of the antibody.

In an embodiment, the antibody disclosed herein is conjugated to a cytotoxic agent, cytostatic agent, toxin, radionuclide, or detectable label. In an embodiment the cytotoxic agent is able to induce death or destruction of a cell in contact therewith. In an embodiment, the cytostatic agent is able to prevent or substantially reduce proliferation and/or inhibits the activity or function of a cell in contact therewith. In an embodiment, the cytotoxic agent or cytostatic agent is a chemotherapeutic agent. In an embodiment, the radionuclide is selected from the group consisting of the isotopes ³H, ¹⁴C, ³²P, ³⁵S, ³⁶Cl, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac, and ¹⁸⁶Re. In an embodiment, the detectable label comprises a fluorescent moiety or a click chemistry handle.

Any immunoglobulin (Ig) constant region can be used in the antibodies disclosed herein. In an embodiment, the Ig region is a human IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂a and IgG₂b) of immunoglobulin molecule.

In an embodiment, one, two, or more mutations (e.g., amino acid substitutions) are introduced into an Fc region (e.g., a CH2 domain (residues 231-340 of human IgG₁)) and/or a CH3 domain (residues 341-447 of human IgG₁, numbered according to the EU numbering system) and/or a hinge region (residues 216-230, numbered according to the EU numbering system) of an antibody described herein, to alter one or more functional properties of the antibody, such as serum half-life, complement fixation, Fc receptor binding, and/or antigen-dependent cellular cytotoxicity.

In an embodiment, one, two, or more mutations (e.g., amino acid substitutions) are introduced into the hinge region of an antibody described herein, such that the number of cysteine residues in the hinge region is altered (e.g., increased or decreased) as described in, e.g., U.S. Pat. No. 5,677,425, herein incorporated by reference in its entirety. The number of cysteine residues in the hinge region may be altered to, e.g., facilitate assembly of the light and heavy chains, or to alter (e.g., increase or decrease) the stability of the antibody.

In an embodiment, one, two, or more amino acid mutations (e.g., substitutions, insertions, or deletions) are introduced into an IgG constant region, or FcRn-binding fragment thereof (preferably an Fc or hinge-Fc fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745, all of which are herein incorporated by reference in their entireties, for examples of mutations that will alter (e.g., decrease or increase) the half-life of an antibody in vivo. In certain embodiments, one, two or more amino acid mutations (e.g., substitutions, insertions, or deletions) are introduced into an IgG constant region, or FcRn-binding fragment thereof (preferably an Fc or hinge-Fc fragment) to decrease the half-life of the antibody in vivo. In other embodiments, one, two or more amino acid mutations (e.g., substitutions, insertions, or deletions) are introduced into an IgG constant region, or FcRn-binding fragment thereof (preferably an Fc or hinge-Fc fragment) to increase the half-life of the antibody in vivo. In an embodiment, the antibodies may have one or more amino acid mutations (e.g., substitutions) in the second constant (CH2) domain (residues 231-340 of human IgG₁) and/or the third constant (CH3) domain (residues 341-447 of human IgG₁), numbered according to the EU numbering system. In an embodiment, the constant region of the IgG₁ of antibody described herein comprises a methionine (M) to tyrosine (Y) substitution in position 252, a serine (S) to threonine (T) substitution in position 254, and a threonine (T) to glutamic acid (E) substitution in position 256, numbered according to the EU numbering system. See U.S. Pat. No. 7,658,921, which is herein incorporated by reference in its entirety. This type of mutant IgG, referred to as “YTE mutant” has been shown to display fourfold increased half-life as compared to wild-type versions of the same antibody (see Dall' Acqua W F et al., (2006) J Biol Chem 281: 23514-24, which is herein incorporated by reference in its entirety). In certain embodiments, an antibody comprises an IgG constant region comprising one, two, three or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into an Fc region (e.g., a CH2 domain (residues 231-340 of human IgG₁) and/or a CH3 domain (residues 341-447 of human IgG₁, numbered according to the EU numbering system) and/or a hinge region (residues 216-230, numbered according to the EU numbering system)) of an antibody described herein, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, all of which are herein incorporated by reference in their entireties.

In an embodiment, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG₁, a variant human IgG₂, or a variant human IgG₄ heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In an embodiment, the FcγRIIB is expressed on a cell selected from the group consisting of macrophages, monocytes, B cells, dendritic cells, endothelial cells, and activated T cells.

In an embodiment, one, two, or more amino acid substitutions are introduced into an IgG constant region Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant region and thereby increase tumor localization. In an embodiment, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276: 6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution; an A330L substitution; an I332E substitution; or a P396L substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, I332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In an embodiment, an antibody described herein comprises the constant region of an IgG₁ with an N297Q or N297A amino acid substitution, numbered according to the EU numbering system. In certain embodiments, an antibody described herein comprises the constant region of an IgG₁ with a mutation selected from the group consisting of D265A, P329A, and a combination thereof, numbered according to the EU numbering system. In another embodiment, an antibody described herein comprises the constant region of an IgG₁ with a mutation selected from the group consisting of L234A, L235A, and a combination thereof, numbered according to the EU numbering system. In another embodiment, an antibody described herein comprises the constant region of an IgG₁ with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG₁ heavy chain, numbered according to the EU numbering system, are not L, L, and D, respectively. This approach is described in detail in International Publication No. WO 14/108483, which is herein incorporated by reference in its entirety. In an embodiment, the amino acids corresponding to positions L234, L235, and D265 in a human IgG₁ heavy chain are F, E, and A; or A, A, and A, respectively, numbered according to the EU numbering system.

In an embodiment, the amino acids at positions 433, 434, and 436 of the heavy chain constant region, according to the EU numbering system, are K, F, and Y, respectively. In an embodiment, the amino acids at positions 252, 254, and 256 of the heavy chain constant region, according to the EU numbering system, are Y, T, and E, respectively. In an embodiment, the amino acids at positions 428 and 434 of the heavy chain constant region, according to the EU numbering system, are L and S, respectively. In an embodiment, the amino acid at positions 309, 311, and 434 of the heavy chain constant region, according to the EU numbering system, are D, H, and S, respectively.

In an embodiment, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In an embodiment, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In an embodiment, the Fc region of an antibody described herein is modified to increase the ability of the antibody to mediate antibody dependent cellular cytotoxicity (ADCC) and/or to increase the affinity of the antibody for an Fey receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In an embodiment, any of the constant region mutations or modifications described herein can be introduced into one or both heavy chain constant regions of an antibody described herein having two heavy chain constant regions.

In an embodiment, the instant disclosure provides an isolated antibody that specifically binds to IL-9 (e.g., human IL-9 or mouse IL-9) and functions as an antagonist (e.g., decreases or inhibits IL-9 activity).

In an embodiment, the instant disclosure provides an isolated antibody that specifically binds to IL-9 (e.g., human IL-9 or mouse IL-9) and decreases or inhibits IL-9 (e.g., human IL-9 or mouse IL-9) activity by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods described herein and/or known to one of skill in the art, relative to IL-9 (e.g., human IL-9 or mouse IL-9) activity without any antibody or with an unrelated antibody (e.g., an antibody that does not specifically bind to IL-9). In an embodiment, the instant disclosure provides an isolated antibody that specifically binds to IL-9 (e.g., human IL-9 or mouse IL-9) and decreases or inhibits IL-9 (e.g., human IL-9 or mouse IL-9) activity by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or more, as assessed by methods described herein and/or known to one of skill in the art, relative to IL-9 (e.g., human IL-9 or mouse IL-9) activity without any antibody or with an unrelated antibody (e.g., an antibody that does not specifically bind to IL-9). Non-limiting examples of IL-9 (e.g., human IL-9 or mouse IL-9) activity can include IL-9 (e.g., human IL-9 or mouse IL-9) signaling, IL-9 (e.g., human IL-9 or mouse IL-9) binding to its receptor (e.g., IL-9Ra); IL-9 (e.g., human IL-9 or mouse IL-9) induced cell proliferation. In an embodiment, a decrease in an IL-9 (e.g., human IL-9 or mouse IL-9) activity is assessed as described in the Examples.

In an embodiment, the instant disclosure provides an isolated antibody that specifically binds IL-9 (e.g., human IL-9 or mouse IL-9) with a dissociation constant (K_{D}) value of less than 10 nM, less than 5 nM, less than 2 nM, less than 1 nM, less than 0.5 nM, or less than 0.1 nM.

### Pharmaceutical Compositions

Provided herein are compositions comprising an isolated anti-IL-9 antibody disclosed herein having the desired degree of purity in a physiologically acceptable carrier, excipient, or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants, including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

In an embodiment, pharmaceutical compositions comprise an isolated anti-IL-9 antibody disclosed herein, and optionally one or more additional prophylactic or therapeutic agents, in a pharmaceutically acceptable carrier. In an embodiment, pharmaceutical compositions comprise an isolated anti-IL-9 antibody herein, and optionally one or more additional prophylactic or therapeutic agents, in a pharmaceutically acceptable carrier. In an embodiment, the antibody is the only active ingredient included in the pharmaceutical composition. In an embodiment, the instant disclosure provides a pharmaceutical composition comprising an isolated anti-IL-9 antibody disclosed herein for use as a medicament. In another embodiment, the instant disclosure provides a pharmaceutical composition for use in a method for the treatment of an inflammatory disease or cancer.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, and Dextrose and Lactated Ringers Injection. Nonaqueous parenteral vehicles include fixed oils of vegetable origin, cottonseed oil, corn oil, sesame oil, and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles, and sodium hydroxide, hydrochloric acid, citric acid or lactic acid for pH adjustment.

A pharmaceutical composition may be formulated for any route of administration to a subject. Specific examples of routes of administration include intranasal, oral, pulmonary, transdermal, intradermal, and parenteral. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol, or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate, and cyclodextrins.

Preparations for parenteral administration of antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof

Topical mixtures comprising an antibody are prepared as described for the local and systemic administration. The resulting mixture can be a solution, suspension, emulsion or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An isolated anti-IL-9 antibody disclosed herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microtine powder for insufflations, alone or in combination with an inert carrier such as lactose. In such a case, the particles of the formulation will, in certain embodiments, have diameters of less than 50 microns, In certain embodiments less than 10 microns.

An isolated anti-IL-9 antibody disclosed herein can be formulated for local or topical application, such as for topical application to the skin and mucous membranes, such as in the eye, in the form of gels, creams, and lotions and for application to the eye or for intracistemal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.

In an embodiment, a pharmaceutical composition comprising antibody described herein is a lyophilized powder, which can be reconstituted for administration as solutions, emulsions, and other mixtures. It may also be reconstituted and formulated as solids or gels. The lyophilized powder is prepared by dissolving antibody described herein, or a pharmaceutically acceptable derivative thereof, in a suitable solvent. In an embodiment, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose, or another suitable agent. The solvent may also contain a buffer, such as citrate, sodium or potassium phosphate, or other such buffer known to those of skill in the art at, in certain embodiments, about neutral pH. Subsequent sterile filtration of the solution followed by lyophilization under standard conditions known to those of skill in the art provides the desired formulation. In an embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The isolated anti-IL-9 antibodies disclosed herein, and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, all of which are herein incorporated by reference in their entireties. In an embodiment, an antibody described herein is targeted to a tumor.

The compositions to be used for in vivo administration can be sterile. This is readily accomplished by filtration through, e.g., sterile filtration membranes.

### Methods of Use and Uses

In an aspect, the instant disclosure provides a method of treating a subject using the anti-IL-9 antibodies disclosed herein. Any disease or disorder in a subject that would benefit from decrease of IL-9 (e.g., human IL-9 or mouse IL-9) function can be treated using the isolated anti-IL-9 antibodies disclosed herein. In an embodiment, the disease or disorder is an inflammatory disease or disorder, an autoimmune disease or disorder, or cancer.

In an embodiment, an inflammatory disease or disorder that can be treated by the methods disclosed herein include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, osteoarthritis, spondyloarthropathies (e.g., psoriatic arthritis, ankylosing spondylitis, Reiter's Syndrome (reactive arthritis), inflammatory osteolysis, Wilson's disease and chronic inflammation resulting from chronic viral or bacterial infections).

In an embodiment, an autoimmune disease or disorder that can be treated by the methods disclosed herein include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

Cancers that can be treated with the isolated anti-IL-9 antibodies or pharmaceutical compositions disclosed herein include, without limitation, a solid tumor, a hematological cancer (e.g., leukemia, lymphoma, myeloma, e.g., multiple myeloma), and a metastatic lesion. In certain embodiments, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, lung cancer (e.g., non-small cell lung cancer or small cell lung cancer), cancer of the small intestine and cancer of the esophagus. The cancer may be at an early, intermediate, late stage, or metastatic cancer.

In an embodiment, the cancer is chosen from lung cancer (e.g., lung adenocarcinoma or non-small cell lung cancer (NSCLC) (e.g., NSCLC with squamous and/or non-squamous histology, or NSCLC adenocarcinoma)), melanoma (e.g., an advanced melanoma), renal cancer (e.g., a renal cell carcinoma), liver cancer (e.g., hepatocellular carcinoma), myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), an ovarian cancer, a colorectal cancer, a pancreatic cancer, a head and neck cancer (e.g., head and neck squamous cell carcinoma (HNSCC)), anal cancer, gastro-esophageal cancer (e.g., esophageal squamous cell carcinoma), mesothelioma, nasopharyngeal cancer, thyroid cancer, cervical cancer, epithelial cancer, peritoneal cancer, or a lymphoproliferative disease (e.g., a post-transplant lymphoproliferative disease).

In an embodiment, the cancer is a hematological cancer, for example, a leukemia, a lymphoma, or a myeloma. In an embodiment, the cancer is a leukemia, for example, acute lymphoblastic leukemia (ALL), acute myelogenous leukemia (AML), acute myeloblastic leukemia (AML), chronic lymphocytic leukemia (CLL), chronic myelogenous leukemia (CML), chronic myeloid leukemia (CML), chronic myelomonocytic leukemia (CMML), chronic lymphocytic leukemia (CLL), or hairy cell leukemia. In an embodiment, the cancer is a lymphoma, for example, B cell lymphoma, diffuse large B-cell lymphoma (DLBCL), activated B-cell like (ABC) diffuse large B cell lymphoma, germinal center B cell (GCB) diffuse large B cell lymphoma, mantle cell lymphoma, Hodgkin lymphoma, non-Hodgkin lymphoma, relapsed non-Hodgkin lymphoma, refractory non-Hodgkin lymphoma, recurrent follicular non-Hodgkin lymphoma, Burkitt lymphoma, small lymphocytic lymphoma, follicular lymphoma, lymphoplasmacytic lymphoma, or extranodal marginal zone lymphoma.

### Polynucleotides, Vectors, and Methods of Producing Antibodies

In an aspect, provided herein are polynucleotides comprising a nucleotide sequence encoding an antibody, or a portion thereof, described herein or a fragment thereof (e.g., a VL and/or VH; and a light chain and/or heavy chain) that specifically binds to an IL-9 (e.g., human IL-9 or mouse IL-9) antigen, and vectors, e.g., vectors comprising such polynucleotides for recombinant expression in host cells (e.g., E. coli and mammalian cells). Provided herein are polynucleotides comprising nucleotide sequences encoding a heavy and/or light chain of an antibody provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular, less than about 10%) of other material, e.g., cellular material, culture medium, other nucleic acid molecules, chemical precursors and/or other chemicals. In an embodiment, a nucleic acid molecule(s) encoding an antibody described herein is isolated or purified.

In an aspect, provided herein are polynucleotides comprising nucleotide sequences encoding antibodies, which specifically bind to an IL-9 (e.g., human IL-9 or mouse IL-9) polypeptide and comprises an amino acid sequence as described herein, as well as antibodies which compete with such antibodies for binding to an IL-9 (e.g., human IL-9 or mouse IL-9) polypeptide (e.g., in a dose-dependent manner), or which binds to the same epitope as that of such antibodies.

In an aspect, provided herein are polynucleotides comprising a nucleotide sequence encoding the light chain or heavy chain of antibody described herein. The polynucleotides can comprise nucleotide sequences encoding a light chain comprising the VL FRs and CDRs of antibodies described herein (see, e.g., Table 2 and Table 3) or nucleotide sequences encoding a heavy chain comprising the VH FRs and CDRs of antibodies described herein (see, e.g., Table 2 and Table 3). In an embodiment, a polynucleotide encodes a VH, VL, heavy chain, and/or light chain of an antibody described herein. In an embodiment, a polynucleotide encodes the first VH and the first VL of an antibody described herein. In an embodiment, a polynucleotide encodes the second VH and the second VL of an antibody described herein. In an embodiment, a polynucleotide encodes the first heavy chain and the first light chain of an antibody described herein. In an embodiment, a polynucleotide encodes the second heavy chain and the second light chain of an antibody described herein. In an embodiment, a polynucleotide encodes the VH and/or the VL, or the heavy chain and/or the light chain, of an isolated antibody described herein.

Also provided herein are polynucleotides encoding an isolated anti-IL-9 antibody that are optimized, e.g., by codon/RNA optimization, replacement with heterologous signal sequences, and elimination of mRNA instability elements. Methods to generate optimized nucleic acids encoding an isolated anti-IL-9 antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In an embodiment, it can be desirable to alter one or more codons to encode a conservative mutation, e.g., a similar amino acid with similar chemical structure and properties and/or function as the original amino acid. Such methods can increase expression of an isolated anti-IL-9 antibody or fragment thereof by at least 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an isolated anti-IL-9 antibody encoded by polynucleotides that have not been optimized.

In an embodiment, an optimized polynucleotide sequence encoding an isolated anti-IL-9 antibody described herein or a fragment thereof (e.g., VL domain and/or VH domain) can hybridize to an antisense (e.g., complementary) polynucleotide of an unoptimized polynucleotide sequence encoding an isolated anti-IL-9 antibody described herein or a fragment thereof (e.g., VL domain and/or VH domain). In an embodiment, an optimized nucleotide sequence encoding an isolated anti-IL-9 antibody described herein or a fragment thereof, hybridizes under high stringency conditions to an antisense polynucleotide of an unoptimized polynucleotide sequence encoding an isolated anti-IL-9 antibody described herein or a fragment thereof. In an embodiment, an optimized nucleotide sequence encoding an isolated anti-IL-9 antibody described herein or a fragment thereof hybridizes under high stringency, intermediate or lower stringency hybridization conditions to an antisense polynucleotide of an unoptimized nucleotide sequence encoding an isolated anti-IL-9 antibody described herein or a fragment thereof. Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding antibodies described herein, e.g., antibodies described in Table 2 and Table 3, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17: 242-6, herein incorporated by reference in its entirety), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antigen-binding region of an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning.

If a clone containing a nucleic acid encoding a particular antigen-binding region or antibody is not available, but the sequence of the antigen-binding region or antibody molecule is known, a nucleic acid encoding the immunoglobulin can be chemically synthesized or obtained from a suitable source (e.g., an antibody cDNA library or a cDNA library generated from, or nucleic acid, preferably poly A+RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding isolated anti-IL-9 (e.g., human IL-9 or mouse IL-9) antibodies described herein can be readily isolated and sequenced using conventional procedures (e.g., by using oligonucleotide probes that are capable of binding specifically to genes encoding the heavy and light chains of the anti-IL-9 (e.g., human IL-9 or mouse IL-9) antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-IL-9 antibodies in the recombinant host cells.

To generate whole antibodies or antigen-binding regions, PCR primers, including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 1 or human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable region, constant regions, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant regions in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In an embodiment, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions is known to those of skill in the art and has been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In an aspect, provided herein are cells (e.g., host cells) expressing (e.g., recombinantly) antibodies described herein which specifically bind to IL-9 (e.g., human IL-9 or mouse IL-9), and related polynucleotides and expression vectors. Provided herein are vectors (e.g., expression vectors) comprising polynucleotides comprising nucleotide sequences encoding anti-IL-9 antibodies or a fragment for recombinant expression in host cells, preferably in mammalian cells (e.g., CHO cells). Also provided herein are host cells comprising such vectors for recombinantly expressing anti-IL-9 antibodies described herein (e.g., human or humanized antibody). In an aspect, provided herein are methods for producing an antibody described herein, comprising expressing the antibody from a host cell.

Recombinant expression of an antibody described herein (e.g., a full-length antigen-binding region or antibody or heavy and/or light chain of an antibody described herein) that specifically binds to IL-9 (e.g., human IL-9 or mouse IL-9) generally involves construction of an expression vector containing a polynucleotide that encodes the antibody. Once a polynucleotide encoding an antibody molecule, heavy and/or light chain of an antibody, or a fragment thereof (e.g., heavy and/or light chain variable regions) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing an antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing an antibody molecule described herein, a heavy or light chain of an antibody, a heavy or light chain variable region of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464, which are herein incorporated by reference in their entireties), and variable regions of the antibody can be cloned into such a vector for expression of the entire heavy, the entire light chain, or both the entire heavy and light chains.

In an embodiment, a vector comprises a polynucleotide encoding a VH, VL, heavy chain, and/or light chain of an antibody described herein. In another embodiment, a vector comprises a polynucleotide encoding the VH and the VL of an antibody described herein. In another embodiment, a vector comprises a polynucleotide encoding the heavy chain and the light chain of an antibody described herein.

An expression vector can be transferred to a cell (e.g., host cell) by conventional techniques and the resulting cells can then be cultured by conventional techniques to produce an antibody described herein or a fragment thereof. Thus, provided herein are host cells containing a polynucleotide encoding containing an antibody described herein or fragments thereof, or a heavy or light chain thereof, or fragment thereof, or a single chain antibody described herein, operably linked to a promoter for expression of such sequences in the host cell.

In an embodiment, a host cell comprises a polynucleotide encoding the VH and VL of an isolated antibody described herein. In another embodiment, a host cell comprises a vector comprising a polynucleotide encoding the VH and VL of an isolated antibody described herein. In another embodiment, a host cell comprises a first polynucleotide encoding the VH of an isolated antibody described herein, and a second polynucleotide encoding the VL of an isolated antibody described herein. In another embodiment, a host cell comprises a first vector comprising a first polynucleotide encoding the VH of an isolated antibody described herein, and a second vector comprising a second polynucleotide encoding the VL of an isolated antibody described herein.

In an embodiment, a heavy chain/heavy chain variable region expressed by a first host cell associated with a light chain/light chain variable region of a second host cell to form an anti-IL-9 (e.g., human IL-9 or mouse IL-9) antibody described herein. In an embodiment, provided herein is a population of host cells comprising such first host cell and such second host cell.

In an embodiment, provided herein is a population of vectors comprising a first vector comprising a polynucleotide encoding a light chain/light chain variable region of an anti-IL-9 (e.g., human IL-9 or mouse IL-9) antibody described herein, and a second vector comprising a polynucleotide encoding a heavy chain/heavy chain variable region of an anti-IL-9 (e.g., human IL-9 or mouse IL-9) antibody described herein.

A variety of host-expression vector systems can be utilized to express antibody molecules described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with, e.g., recombinant bacteriophage DNA, plasmid DNA, or cosmid DNA expression vectors containing antibody coding sequences; yeast (e.g., Saccharomyces and Pichia) transformed with, e.g., recombinant yeast expression vectors containing antibody coding sequences; insect cell systems infected with, e.g., recombinant virus expression vectors (e.g., baculovirus) containing antibody coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In an embodiment, cells for expressing antibodies described herein are Chinese hamster ovary (CHO) cells, for example CHO cells from the CHO GS System™ (Lonza). In an embodiment, the heavy chain and/or light chain of an antibody produced by a CHO cell may have an N-terminal glutamine or glutamate residue replaced by pyroglutamate. In an embodiment, cells for expressing antibodies described herein are human cells, e.g., human cell lines. In an embodiment, a mammalian expression vector is pOptiVEC™ or pcDNA3.3. In an embodiment, bacterial cells such as Escherichia coli, or eukaryotic cells (e.g., mammalian cells), especially for the expression of whole recombinant antibody molecule, are used for the expression of a recombinant antibody molecule. For example, mammalian cells such as CHO cells, in conjunction with a vector such as the major intermediate early gene promoter element from human cytomegalovirus is an effective expression system for antibodies (Foecking M K & Hofstetter H (1986) Gene 45: 101-5; and Cockett M I et al., (1990) Biotechnology 8(7): 662-7, each of which is herein incorporated by reference in its entirety). In an embodiment, antibodies described herein are produced by CHO cells or NS0 cells. In an embodiment, the expression of nucleotide sequences encoding antibodies described herein which specifically bind to IL-9 (e.g., human IL-9 or mouse IL-9) is regulated by a constitutive promoter, inducible promoter, or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (e.g., see Logan J & Shenk T (1984) PNAS 81(12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544, which is herein incorporated by reference in its entirety).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NSO (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In an embodiment, anti-IL-9 (e.g., human IL-9 or mouse IL-9) antibodies described herein are produced in mammalian cells, such as CHO cells.

In an embodiment, the antibodies described herein have reduced fucose content or no fucose content. Such antibodies can be produced using techniques known one skilled in the art. For example, the antibodies can be expressed in cells deficient or lacking the ability of to fucosylate. In an example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express an anti-IL-9 (e.g., human IL-9 or mouse IL-9) antibody described herein can be engineered. In an embodiment, a cell provided herein stably expresses a light chain/light chain variable region and a heavy chain/heavy chain variable region which associate to form an antigen-binding region, or an antibody described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for one to two days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-IL-9 (e.g., human IL-9 or mouse IL-9) described herein or a fragment thereof. Such engineered cell lines can be particularly useful in the screening and evaluation of compositions that interact directly or indirectly with the antibody molecule.

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77(6): 3567-70; O′Hare K et al., (1981) PNAS 78: 1527-31); gpt, which confers resistance to mycophenolic acid (Mulligan RC & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150: 1-14, all of which are herein incorporated by reference in their entireties.

The expression levels of an antibody molecule can be increased by vector amplification (for a review, see, Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system is amplifiable, increase in the level of inhibitor present in culture of host cell will increase the number of copies of the marker gene. Since the amplified region is associated with the gene of interest, production of the protein will also increase (Crouse G F et al., (1983) Mol Cell Biol 3: 257-66, which is herein incorporated by reference in its entirety).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot NJ (1986) Nature 322: 562-565; and Köhler G (1980) PNAS 77: 2197-2199, each of which is herein incorporated by reference in its entirety). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more genes/nucleotide sequences, or in the range of 2-5, 5-10, or 10-20 genes/nucleotide sequences. For example, a bicistronic nucleic acid construct can comprise, in the following order, a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In an embodiment, an antibody described herein is isolated or purified. In an embodiment, an isolated antibody is one that is substantially free of other antibodies with different antigenic specificities than the isolated antibody. For example, in certain embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In an embodiment, antibodies described herein are isolated or purified.

Anti-IL-9 (e.g., human IL-9 or mouse IL-9) antibodies or fragments thereof can be produced by any method known in the art for the synthesis of proteins or antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In an embodiment, an antibody described herein is prepared, expressed, created, or isolated by any means that involves creation, e.g., via synthesis, genetic engineering of DNA sequences. In certain embodiments, such an antibody comprises sequences (e.g., DNA sequences or amino acid sequences) that do not naturally exist within the antibody germline repertoire of an animal or mammal (e.g., human) in vivo.

In one aspect, provided herein is a method of making an anti-IL-9 (e.g., human IL-9 or mouse IL-9) antibody comprising culturing a cell or host cell described herein. In an embodiment, the method is performed in vitro. In an aspect, provided herein is a method of making an anti-IL-9 (e.g., human IL-9 or mouse IL-9) antibody comprising expressing (e.g., recombinantly expressing) the antibody using a cell or host cell described herein (e.g., a cell or a host cell comprising polynucleotides encoding an antibody described herein). In an embodiment, the cell is an isolated cell. In an embodiment, the exogenous polynucleotides have been introduced into the cell. In an embodiment, the method further comprises the step of purifying the antibody obtained from the cell or host cell.

In an embodiment, an isolated antibody is produced by expressing in a cell a polynucleotide encoding the VH and VL of an antibody described herein under suitable conditions so that the polynucleotides are expressed, and the antibody is produced. In another embodiment, an isolated antibody is produced by expressing in a cell a polynucleotide encoding the heavy chain and light chain of an antibody described herein under suitable conditions so that the polynucleotides are expressed, and the antibody is produced. In an embodiment, an isolated antibody is produced by expressing in a cell a first polynucleotide encoding the VH of an antibody described herein, and a second polynucleotide encoding the VL of an antibody described herein, under suitable conditions so that the polynucleotides are expressed, and the antibody is produced. In an embodiment, an isolated antibody is produced by expressing in a cell a first polynucleotide encoding the heavy chain of an antibody described herein, and a second polynucleotide encoding the light chain of an antibody described herein, under suitable conditions so that the polynucleotides are expressed, and the antibody is produced.

Methods for producing polyclonal antibodies are known in the art (see, for example, Chapter 11 in: Short Protocols in Molecular Biology, (2002) 5th Ed., Ausubel F M et al., eds., John Wiley and Sons, New York, which is herein incorporated by reference in its entirety).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), each of which is herein incorporated by reference in its entirety. The term “monoclonal antibody” as used herein is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In an embodiment, a “monoclonal antibody,” as used herein, is an antibody produced by a single cell (e.g., hybridoma or host cell producing a recombinant antibody), wherein the antibody specifically binds to anti-IL-9 (e.g., human IL-9 or mouse IL-9) as determined, e.g., by ELISA or other antigen-binding or competitive binding assay known in the art or in the examples provided herein. In an embodiment, a monoclonal antibody can be a chimeric antibody or a humanized antibody. In an embodiment, a monoclonal antibody is a monovalent antibody or multivalent (e.g., bivalent) antibody. In an embodiment, a monoclonal antibody is a monospecific or multispecific antibody (e.g., bispecific antibody). Monoclonal antibodies described herein can, for example, be made by the hybridoma method as described in Kohler G & Milstein C (1975) Nature 256: 495, which is herein incorporated by reference in its entirety, or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed Ausubel F M et al., supra).

As used herein, an antibody binds to an antigen multivalently (e.g., bivalently) when the antibody comprises at least two (e.g., two or more) monovalent binding regions, each monovalent binding region capable of binding to an epitope on the antigen. Each monovalent binding region can bind to the same or different epitopes on the antigen.

Methods for producing and screening for specific antibodies using hybridoma technology are routine and well known in the art. For example, in the hybridoma method, a mouse or other appropriate host animal, such as a sheep, goat, rabbit, rat, hamster, or macaque monkey, is immunized to elicit lymphocytes that produce or are capable of producing antibodies that will specifically bind to the protein used for immunization (e.g., IL-9). Alternatively, lymphocytes may be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-103 (Academic Press, 1986), herein incorporated by reference in its entirety). Additionally, a RIMMS (repetitive immunization multiple sites) technique can be used to immunize an animal (Kilpatrick K E et al., (1997) Hybridoma 16:381-9, herein incorporated by reference in its entirety).

In an embodiment, mice (or other animals, such as rats, monkeys, donkeys, pigs, sheep, hamster, or dogs) can be immunized with an antigen (e.g., IL-9) and once an immune response is detected, e.g., antibodies specific for the antigen are detected in the mouse serum, the mouse spleen is harvested and splenocytes isolated. The splenocytes are then fused by well-known techniques to any suitable myeloma cells, for example, cells from cell line SP20 available from the American Type Culture Collection (ATCC®) (Manassas, VA), to form hybridomas. Hybridomas are selected and cloned by limited dilution. In an embodiment, lymph nodes of the immunized mice are harvested and fused with NSO myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In an embodiment, myeloma cells are employed that fuse efficiently, support stable high-level production of antibody by the selected antibody-producing cells, and are sensitive to a medium such as HAT medium. Among these myeloma cell lines are murine myeloma lines, such as the NSO cell line or those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor D (1984) J Immunol 133: 3001-5; Brodeur et al., Monoclonal Antibody Production Techniques and Applications, pp. 51-63 (Marcel Dekker, Inc., New York, 1987), each of which is herein incorporated by reference in its entirety).

Culture medium in which hybridoma cells are growing is assayed for production of monoclonal antibodies directed against IL-9 (e.g., human IL-9 or mouse IL-9). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding J W (Ed), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include, e.g., antibody fragments which recognize IL-9 (e.g., human IL-9 or mouse IL-9), and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antigen-binding region that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108, all of which are herein incorporated by reference in their entireties.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax R L et al., (1992) BioTechniques 12(6): 864-9; Sawai H et al., (1995) Am J Reprod Immunol 34: 26-34; and Better M et al., (1988) Science 240: 1041-1043, all of which are herein incorporated by reference in their entireties.

In certain embodiments, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison S L (1985) Science 229: 1202-7; Oi V T & Morrison S L (1986) BioTechniques 4: 214-221; Gillies S D et al., (1989) J Immunol Methods 125: 191-202; and U.S. Pat. Nos. 5,807,715, 4,816,567, 4,816,397 and 6,331,415, all of which are herein incorporated by reference in their entireties.

A humanized antibody is capable of binding to a predetermined antigen, and which comprises a framework region having substantially the amino acid sequence of a human immunoglobulin and CDRs having substantially the amino acid sequence of a non-human immunoglobulin (e.g., a murine immunoglobulin). In certain embodiments, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA, and IgE, and any isotype, including IgG₁, IgG₂, IgG₃, and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28(4/5): 489-498; Studnicka G M et al., (1994) Prot Engineering 7(6): 805-814; and Roguska M A et al., (1994) PNAS 91: 969-973), chain shuffling (U.S. Pat. No. 5,565,332), and techniques disclosed in, e.g. , U.S. Pat. Nos. 6,407,213, 5,766,886, International Publication No. WO 93/17105; Tan P et al., (2002) J Immunol 169: 1119-25; Caldas C et al., (2000) Protein Eng. 13(5): 353-60; Morea V et al., (2000) Methods 20(3): 267-79; Baca M et al., (1997) J Biol Chem 272(16): 10678-84; Roguska M A et al., (1996) Protein Eng 9(10): 895 904; Couto J R et al., (1995) Cancer Res. 55 (23 Supp): 5973s-5977s; Couto J R et al., (1995) Cancer Res 55(8): 1717-22; Sandhu J S (1994) Gene 150(2): 409-10 and Pedersen JT et al., (1994) J Mol Biol 235(3): 959-73, all of which are herein incorporated by reference in their entireties. See also, U.S. Application Publication No. US 2005/0042664 A1 (Feb. 24, 2005), which is herein incorporated by reference in its entirety.

Methods for making multispecific antibodies (e.g., bispecific antibodies) have been described, see, for example, U.S. Pat. Nos. 7,951,917; 7,183,076; 8,227,577; 5,837,242; 5,989,830; 5,869,620; 6,132,992 and 8,586,713, all of which are herein incorporated by reference in their entireties.

Bispecific, bivalent antibodies, and methods of making them, are described, for instance in U.S. Pat. Nos. 5,731,168, 5,807,706, 5,821,333, and U.S. Appl. Publ. Nos. 2003/020734 and 2002/0155537; each of which is herein incorporated by reference in its entirety. Bispecific tetravalent antibodies, and methods of making them are described, for instance, in Int. Appl. Publ. Nos. WO 02/096948 and WO 00/44788, the disclosures of both of which are herein incorporated by reference in its entirety. See generally, Int. Appl. Publ. Nos. WO 93/17715, WO 92/08802, WO 91/00360, and WO 92/05793; Tuft et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

A bispecific antibody as described herein can be generated according to the DuoBody technology platform (Genmab A/S) as described, e.g., in International Publication Nos. WO 2011/131746, WO 2011/147986, WO 2008/119353, and WO 2013/060867, and in Labrijn A F et al., (2013) PNAS 110(13): 5145-5150. The DuoBody technology can be used to combine one half of a first monospecific antibody, or first antigen-binding region, containing two heavy and two light chains with one half of a second monospecific antibody, or second antigen-binding region, containing two heavy and two light chains. The resultant heterodimer contains one heavy chain and one light chain from the first antibody, or first antigen-binding region, paired with one heavy chain and one light chain from the second antibody, or second antigen-binding region. When both of the monospecific antibodies, or antigen-binding regions, recognize different epitopes on different antigens, the resultant heterodimer is a bispecific antibody.

The DuoBody technology requires that each of the monospecific antibodies, or antigen-binding regions includes a heavy chain constant region with a single point mutation in the CH3 domain. The point mutations allow for a stronger interaction between the CH3 domains in the resultant bispecific antibody than between the CH3 domains in either of the monospecific antibodies, or antigen-binding regions. The single point mutation in each monospecific antibody, or antigen-binding region, is at residue 366, 368, 370, 399, 405, 407, or 409, numbered according to the EU numbering system, in the CH3 domain of the heavy chain constant region, as described, e.g., in International Publication No. WO 2011/131746. Moreover, the single point mutation is located at a different residue in one monospecific antibody, or antigen-binding region, as compared to the other monospecific antibody, or antigen-binding region. For example, one monospecific antibody, or antigen-binding region, can comprise the mutation F405L (i.e., a mutation from phenylalanine to leucine at residue 405), while the other monospecific antibody, or antigen-binding region, can comprise the mutation K409R (i.e., a mutation from lysine to arginine at residue 409), numbered according to the EU numbering system. The heavy chain constant regions of the monospecific antibodies, or antigen-binding regions, can be an IgG₁, IgG₂, IgG₃, or IgG₄ isotype (e.g., a human IgG₁ isotype), and a bispecific antibody produced by the DuoBody technology can retain Fc-mediated effector functions.

Another method for generating bispecific antibodies has been termed the “knobs-into-holes” strategy (see, e.g., Intl. Publ. WO2006/028936). The mispairing of Ig heavy chains is reduced in this technology by mutating selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In some embodiments, compositions of the invention have immunoglobulin chains in which the CH3 domains have been modified by mutating selected amino acids that interact at the interface between two polypeptides so as to preferentially form a bispecific antibody. The bispecific antibodies can be composed of immunoglobulin chains of the same subclass (e.g., IgG₁ or IgG₃) or different subclasses (e.g., IgG₁ and IgG₃, or IgG₃ and IgG₄).

Bispecific antibodies can, in some instances contain, IgG₄ and IgG₁, IgG₄ and IgG₂, IgG₄ and IgG₂, IgG₄ and IgG₃, or IgG₁ and IgG³ chain heterodimers. Such heterodimeric heavy chain antibodies can routinely be engineered by, for example, modifying selected amino acids forming the interface of the CH3 domains in human IgG₄ and the IgG₁ or IgG₃ so as to favor heterodimeric heavy chain formation.

In an embodiment, an antibody described herein, which binds to the same epitope of IL-9 (e.g., human IL-9 or mouse IL-9) as an anti-IL-9 antibody described herein, is a human antibody. In an embodiment, an antibody described herein, which competitively blocks (e.g., in a dose-dependent manner) any one of the antibodies described herein, from binding to IL-9 (e.g., human IL-9 or mouse IL-9), is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., IL-9). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg N & Huszar D (1995) Int Rev Immunol 13:65-93, herein incorporated by reference in its entirety. For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318 and 5,939,598, all of which are herein incorporated by reference in their entireties. Examples of mice capable of producing human antibodies include the Xenomouse™ (Abgenix, Inc.; U.S. Pat. Nos. 6,075,181 and 6,150,184), the HuAb-Mouse™ (Medarex, Inc./Gen Pharm; U.S. Pat. Nos. 5,545,806 and 5,569, 825), the Trans Chromo Mouse™ (Kirin) and the KM Mouse™ (Medarex/Kirin), all of which are herein incorporated by reference in their entireties.

Human antibodies that specifically bind to IL-9 (e.g., human IL-9 or mouse IL-9) can be made by a variety of methods known in the art including the phage display methods described above using antibody libraries derived from human immunoglobulin sequences. See also, U.S. Pat. Nos. 4,444,887, 4,716,111 and 5,885,793; and International Publication Nos. WO 98/46645, WO 98/50433, WO 98/24893, WO 98/16654, WO 96/34096, WO 96/33735, and WO 91/10741, all of which are herein incorporated by reference in their entireties.

In certain embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., IL-9). Such methods are known and are described in the art, see, e.g., Shinmoto H et al., (2004) Cytotechnology 46: 19-23; Naganawa Y et al., (2005) Human Antibodies 14: 27-31, each of which is herein incorporated by reference in its entirety.

### Kits

Also provided are kits comprising one or more antibodies described herein, or pharmaceutical compositions or conjugates thereof. In an embodiment, provided herein is a pharmaceutical pack or kit comprising one or more containers filled with one or more of the ingredients of the pharmaceutical compositions described herein, such as one or more antibodies provided herein. In an embodiment, the kits contain a pharmaceutical composition described herein and any prophylactic or therapeutic agent, such as those described herein. In an embodiment, the kits may contain a T cell mitogen, such as, e.g., phytohaemagglutinin (PHA) and/or phorbol myristate acetate (PMA), or a TCR complex stimulating antibody, such as an anti-CD3 antibody and anti-CD28 antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided, are kits that can be used in the above methods. In an embodiment, a kit comprises an antibody described herein, preferably purified antibody, in one or more containers. In an embodiment, kits described herein contain a substantially isolated IL-9 (e.g., human IL-9 or mouse IL-9) antigen as a control. In an embodiment, the kits described herein further comprise a control antibody which does not react with IL-9 (e.g., human IL-9 or mouse IL-9) antigen. In an embodiment, kits described herein contain one or more elements for detecting the binding of an antibody to an IL-9 (e.g., human IL-9 or mouse IL-9) antigen (e.g., the antibody can be conjugated to a detectable substrate such as a fluorescent compound, an enzymatic substrate, a radioactive compound, or a luminescent compound, or a second antibody which recognizes the first antibody can be conjugated to a detectable substrate). In an embodiment, a kit provided herein can include a recombinantly produced or chemically synthesized IL-9 (e.g., human IL-9 or mouse IL-9) antigen. The IL-9 (e.g., human IL-9 or mouse IL-9) antigen provided in the kit can also be attached to a solid support. In an embodiment, the detecting means of the above-described kit includes a solid support to which an IL-9 (e.g., human IL-9 or mouse IL-9) antigen is attached. Such a kit can also include a non-attached reporter-labeled anti-human antibody or anti-mouse/rat antibody. In this embodiment, binding of the antibody to the IL-9 (e.g., human IL-9 or mouse IL-9) antigen can be detected by binding of the said reporter-labeled antibody. In certain embodiments, the present invention relates to the use of a kit of the present invention for in vitro assaying and/or detecting IL-9 (e.g., human IL-9 or mouse IL-9) antigen in a biological sample.

### EXAMPLES

The examples in this Section(i.e., Section 6) are offered by way of illustration and not by way of limitation.

### Example 1: Generation of Neutralizing IL-9 Monoclonal Antibodies

### A. Llama Immunization and Library Construction:

Llama, farmed outdoors according to the French animal welfare legislation, were immunized intramuscularly with recombinant human IL-9 or mouse IL-9 (R&D Systems) and boosted weekly for six weeks. Briefly, each llama (4 in total) received 40 μg of IL-9, buffered in phosphate-buffered saline (PBS) and mixed with Incomplete Freund's Adjuvant (Sigma-Aldrich) for the first two weeks, and 20 μg of IL-9 for the remaining four weeks. Generation of Fab libraries was performed using the SIMPLE antibody platform as previously described (see WO2010/001251, the contents of which are incorporated herein in their entirety). Five days after the last immunization, 400 mL of blood containing peripheral blood lymphocytes was collected from the llamas, purified by centrifugation on a Ficoll-Paque gradient and used for extraction of total RNA. Total RNA was then converted into random primed cDNA using reverse transcriptase, and gene sequences encoding for VH-CH1 regions of llama IgG1 and VL-CL domains (kappa and lambda) were isolated by PCR and subcloned into a phagemid vector pCB3. The pCB3 vector allows expression of recombinant antibodies as Fab fragments fused to the phage pIII envelope protein.

### B. Selection of Fabs Binding to IL-9:

The E. coli strain TG1 (Netherlands Culture Collection of Bacteria) was transformed using recombinant phagemids to generate Fab-expressing phage libraries (one lambda and one kappa library per immunized llama). The resulting Fab-expressing phages, having a diversity in the range of 108-109, were then adsorbed on immobilized recombinant biotinylated IL-9, and eluted using trypsin as previously described (De Haard et al. (1999) Journal of Biological Chemistry, 274: 18218-30). Three rounds of selections were performed to enrich for phages expressing IL-9-specific Fabs. TG1 E. coli was finally infected with selected phages, and individual colonies were isolated. Secretions of Fabs into periplasm of E. coli strain TG1 were induced using isopropyl β-D-1-thiogalactopyranoside (Sigma-Aldrich) under low glucose concentrations (0.1% w/v) and the Fab-containing periplasmic fractions of bacteria were collected.

### C. Fab Screening, Characterization and Production:

The binding of Fabs (periplasmic extract) to their respective mouse or human targets was determined by surface plasmon resonance (SPR) using a Biacore 3000 apparatus (GE Healthcare). IL-9 was immobilized on a carboxymethyl dextran sensor chip (CM-5) using amine coupling in sodium acetate buffer (GE Healthcare). The Fab-containing periplasmic extracts were loaded with a flow rate of 30 μ/min. The Fab binding and off-rates were measured over a 90 second period (Table 5). Binding clones were sequenced and VHs were grouped in families. Out of this selection, 11 different families were identified against human IL-9, and 10 families were identified against mouse IL-9. Furthermore, the ability of the antibodies to compete for human or mouse IL-9 binding to human or rat IL-9R was tested also in a Biacore 3000. For this assay, human or rat IL-9R was coated at high density on a carboxymethyl dextran sensor chip (CM-5). Then, a premade mixture of periplasmic extract (Fab) and IL-9 was injected. The non-binding of IL-9 to the coated receptors indicated that the binding of the Fab was competing with IL-9R binding.

TABLE 5
Off rate of Fabs from periplasmic extracts.

| Clone | BIAcore - Kd human IL9 (s⁻¹) | BIAcore - Kd mouse IL9 (s⁻¹) |
|---|---|---|
| 7D6 | 8.4E−04 | n.b. |
| 8C3 | 6.7E−04 | n.b. |
| 6C4 | 4.4E−04 | n.b. |
| 6E2 | 2.8E−04 | n.b. |
| 7A4 | 1.3E−04 | n.b. |
| 6D3 | 1.7E−04 | n.b. |
| 35D8 | n.b. | 4.70E−04 |

(n.b. = no binding)

### D. Monospecific Ab Production, Purification and Characterization:

The cDNAs encoding the VH and VL (lambda or kappa) domains of the eight most potent (those with the lowest k_{off} s⁻¹: 7D6, 8C3, 6C4, 6E2, 7A4, 6D3, 6F2, and 8G3), neutralizing hIL-9-specific Fabs, from different VH families, were selected and re-engineered as full IgGs. The full IgGs were cloned into two separate pUPE mammalian expression vectors, one comprising the cDNAs encoding the CH1, CH2, and CH3 domains of human IgG1 containing mutations that abrogate Ab effector functions mediated by the Fc receptor, and the other comprising the CL domain (lambda or kappa). For the anti-mIL-9 Fabs, only the most potent one VH and VL (35D8) was recloned as a full mIgG2a. The antibodies were produced by transient transfection of mammalian cells and purified by protein A affinity chromatography, as previously described (Basilico et al. (2014) Journal of Clinical Investigation 124:3172).

The CDR, VH and VL sequences of the selected antibodies are shown in Tables 2-4 above.

### Example 2: In Vitro Characterization of IL-9 mAbs

The IL-9 mAbs were tested for their ability to bind to their respective targets in vitro and to inhibit cellular effects mediated by IL-9 signalling.

### A. Inhibition of IL-9-Induced Baf3hIL9RA6 Cell Proliferation:

The neutralizing activity of the IL-9 mAbs was assessed in in vitro cellular assays, using Baf3hIL9RA6 cells, which proliferate in response to IL-9.

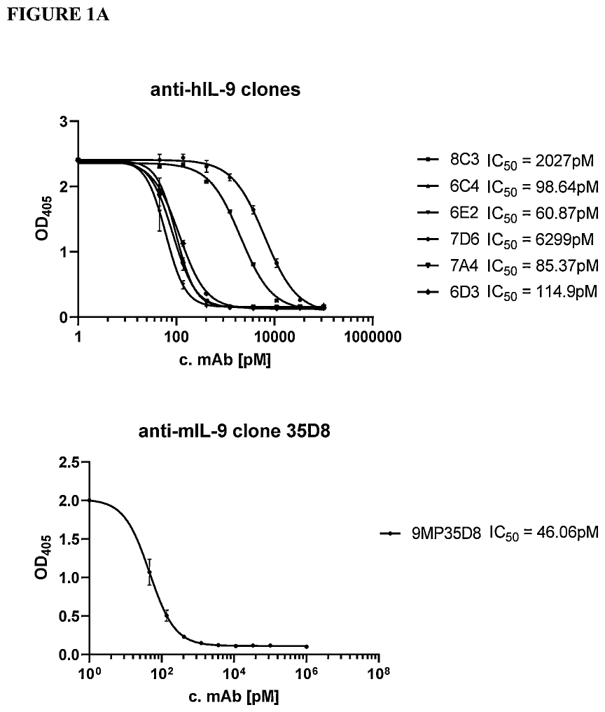

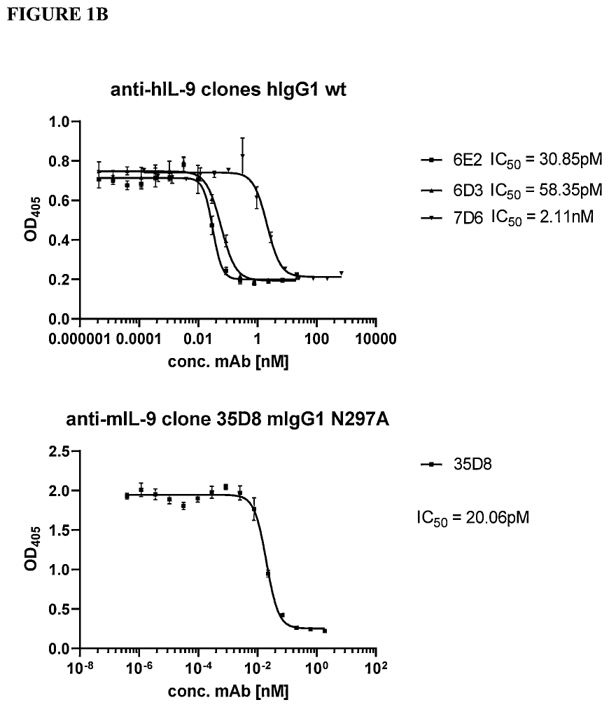

Human IL-9 SN baculo (50 U/mL) was incubated with eight different concentrations of the IL-9 mAbs (ng/ml) for 30 minutes. Then, 3,000 Baf3h9RA6 cells were added and after 3 days the hexosaminidase substrate was added for 2 hours and 30 minutes and hexosaminidase activity was measured. As shown in FIG. 1A, IL-9 specific mAbs potently inhibited human IL-9-induced Baf3hIL9RA6 cell proliferation. Specifically, hIL-9 mAbs blocked the cellular proliferation induced by human IL-9 with an IC50 from 61 pM to 6.3 nM. To confirm the high potency of some antibodies, new batches of the antibodies were produced and retested (FIG. 1B). The high potency of anti-hIL9 antibody 6E2 and 6D3 (IC50 of 30.85 pM and 58.35 pM, respectively) was confirmed while the lower potency of 7D6 was also confirmed (2.11 nM).

The mouse IL-9 mAb was also tested for potency in vitro. Mouse IL-9 SN baculo (20U/ml) was incubated with eight different concentrations of anti-IL9 (ng/ml) for 30 minutes. Then, 3,000 TS1 cells were added and after 3 days the hexosaminidase activity was measured. Substrate of hexosaminidase was added for 2 h30 before the measure. As shown in FIG. 1A, antibody 35D8 neutralized mIL9 with a potency of 46 pM. The very high potency of the mIL9 antibody was confirmed with a new batch of antibody (mIgG1-N297A) with an IC50 of 20.06 pM (FIG. 1B).

B. Affinity of Neutralizing IL-9 mAbs

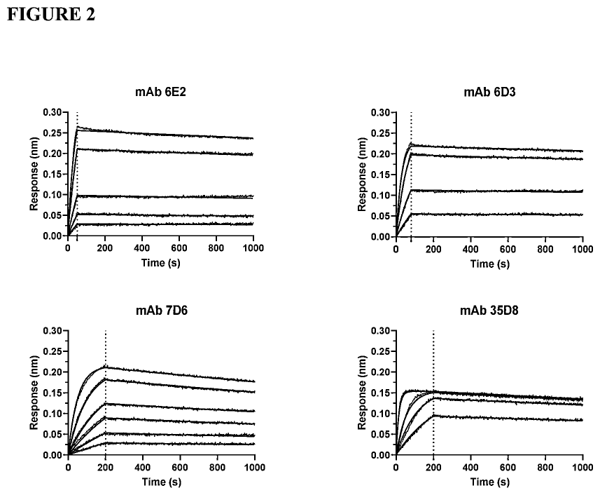

Bio-layer interferometry (BLI) experiments were used to analyze the affinity of three anti-hIL-9 antibodies (6E2, 6D3, and 7D6) and one anti-mIL-9 antibody (35D8) (FIG. 2). The BLI experiments on the antagonistic antibodies were performed using an Octet Red 96 machine (Sartorius) in kinetics buffer (PBS, 0.1% (w/v) BSA, 0.02% (v/v) Tween20) at 298 K. Anti-hIgG Fc capture (AHC) or anti-mIgG Fc capture sensors (Sartorius) were functionalized with the IL-9 mAbs. The functionalized tips were then dipped in different concentrations of hIL-9 or mIL-9 (both from R&D systems). Non-functionalized tips were used as negative controls in a double referenced setup. After subtracting the control sensorgrams, the resulting data was fitted with a 1:1 binding model. Data analysis was performed using the Data Analysis software 9.0.0.14 (Sartorius) (Table 6). The results show that the anti-hIL-9 antibodies had a strong affinity for hIL-9, and that the anti-mIL-9 antibody, 35D8, had a strong affinity for mIL-9.

TABLE 6
Kinetics properties of the IL-9 mAbs.

| | | | BLI | | | | | | Bioassay |
|---|---|---|---|---|---|---|---|---|---|
| Clone | Target | MoA | kₐ [1/Ms] | kₐ error | k_{d} [1/s] | k_{d} error | K_{D} [nM] | K_{D} error [nM] | IC₅₀ [pM] |
| 6E2 | hIL-9 | neutr. | 3.34E+05 | 4.13E+03 | 7.88E−05 | 9.29E−07 | 0.24 | 0.004 | 30.85 |
| 6D3 | hIL-9 | neutr. | 4.27E+05 | 2.42E+03 | 5.72E−05 | 8.74E−07 | 0.13 | 0.002 | 58.35 |
| 7D6 | hIL-9 | neutr. | 1.24E+05 | 4.83E+02 | 2.23E−04 | 1.21E−06 | 1.81 | 0.012 | 2111 |
| 35D8 | mIL-9 | neutr. | 1.39E+06 | 6.90E+03 | 1.59E−04 | 1.41E−06 | 0.11 | 0.001 | 20.06 |

### Example 3: In Vivo Characterization of Anti-IL-9 mAbs in a Murine Model of Asthma

Patients with acute asthma that suffer from uncontrollable disease despite being on maximum corticosteroid therapy require further treatment with biologics. Type 2 helper T cells (Th2) are the key cell type that drive asthma pathology. However, it is believed that besides Th2 cells, their innate counterparts, namely group 2 innate lymphocytes (ILC2), also play a profound role in acute asthmatics, especially due to their steroid resistance.

ILC2s are characterized by high expression of IL-9, which has been shown to promote proliferation of ILC2s in an autocrine mechanism. Thus, blocking IL-9 with antibodies could provide a solution for patients suffering from ILC2-driven acute asthma.

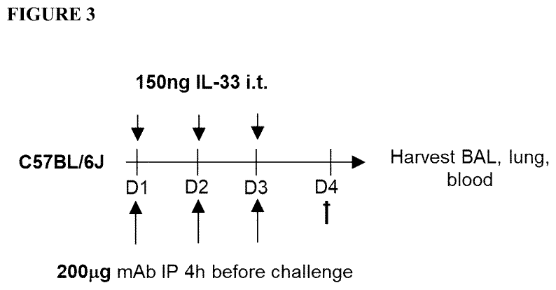

An ILC2/IL-9 dependent murine asthma model is used to test the antibodies in-vivo. In this murine model, recombinant IL-33 is administered to the lungs of C57BL/6J mice to activate ILC2s (Du et al, 2020). The anti-IL-9 mAbs are administered intraperitoneally at a dose of 200 μg, for three consecutive days (n=6 per group) (FIG. 3). On the same days, mice are slightly anesthetized with isoflurane (2.5% in air) and challenged with 150 ng of rIL-33 intratracheally. The IL-33 challenges are performed at least 4 hours after anti-IL9 mAb administrations to assure sufficient biodistribution of the mAbs. On day 4, mice are sacrificed and bronchoalveolar lavage (BAL) is performed, and complete lungs are isolated. The isolated lungs are digested in RPMI containing 10% FCS+liberase 1/50 and DNAse 1/1000 to yield single cell suspensions. Cell compartments of bronchoalveolar lavage (BAL) fluid and lung samples are further analyzed by flow cytometry, using a flow panel designed to analyse eosinophilia and ILC2 activation markers.

### Example 4: Structural Analysis of the Binding Anti-IL9 Antibodies

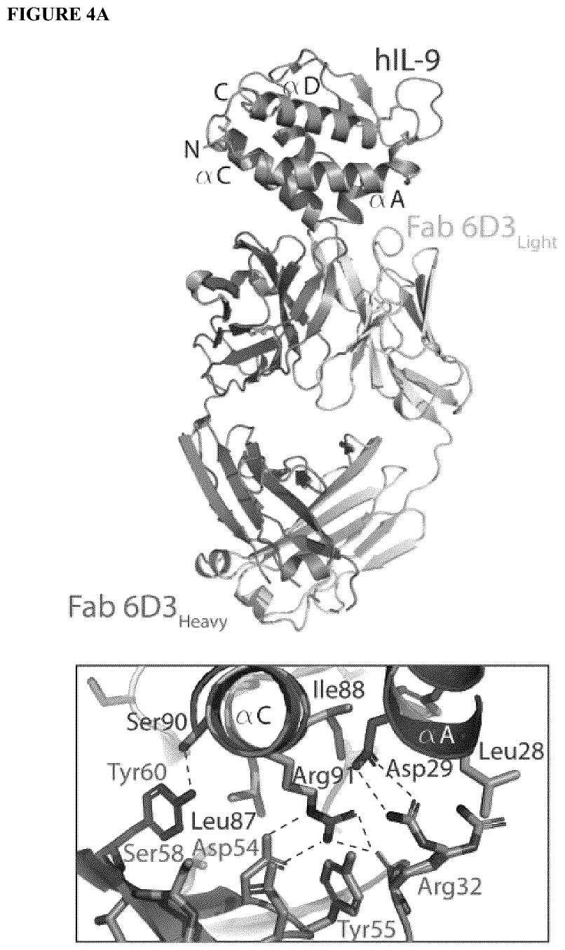

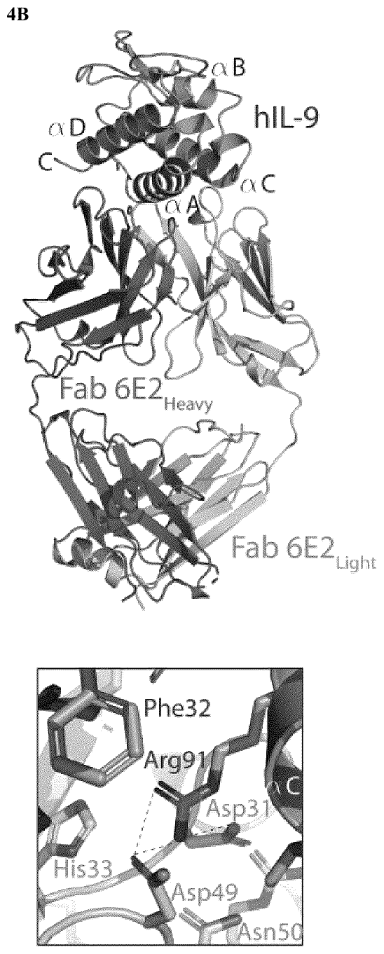

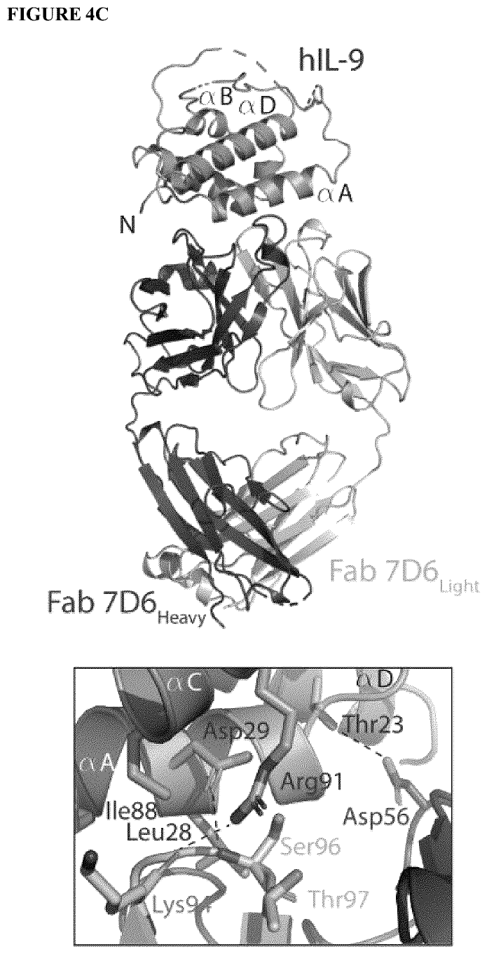

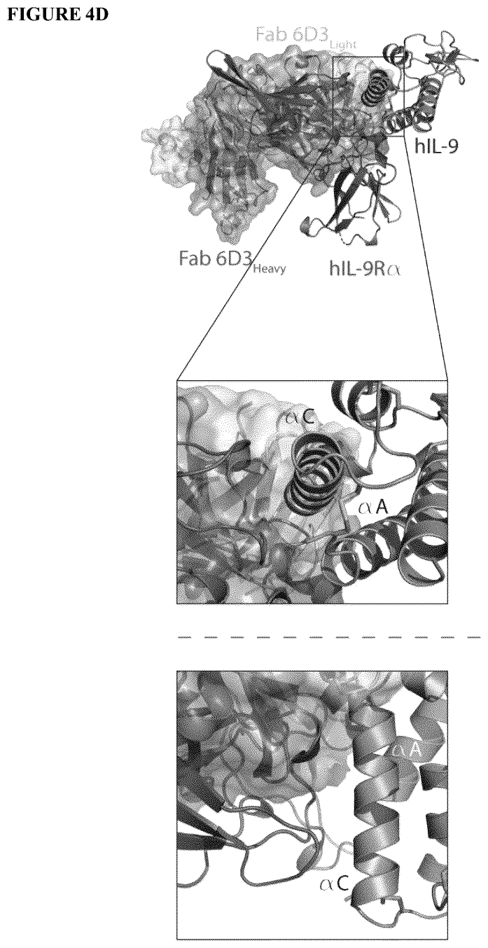

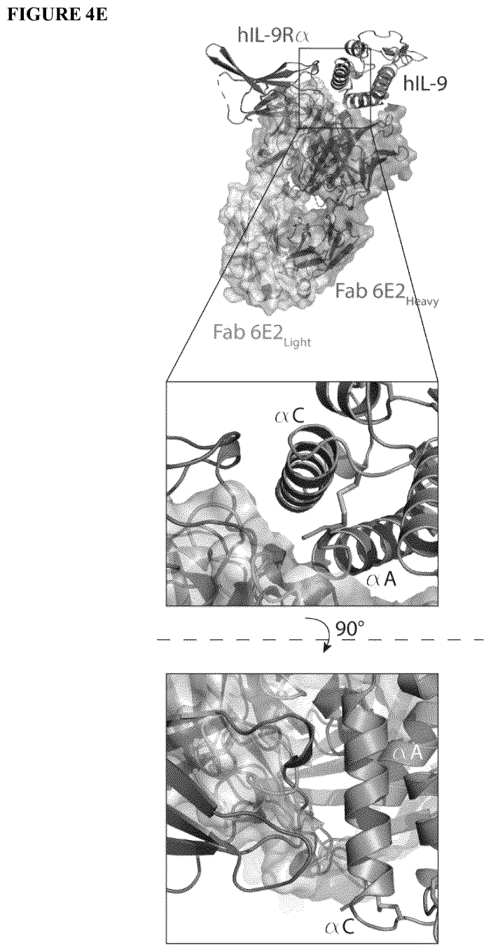

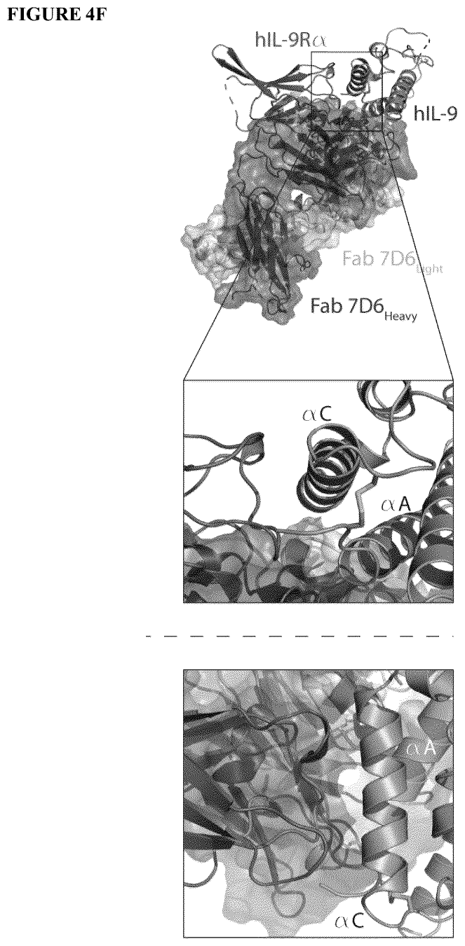

The structure of the Fab:IL-9 complexes were determined by X-ray crystallography. The first Fab:hIL-9 complex (Fab 6D3:hIL-9) crystal resulted in a dataset with a resolution of 1.7 A (Table 7). The resulting map, after phasing by molecular replacement using a model for the Fab, allowed hIL-9 to be built de novo in the electron density without any predispositions. In terms of the Fab:hIL-9 interface, Fab 6D3 targets hIL-9 by mainly binding the C-helix and the first half of the A-helix with a polar footprint covering an interface area of 750.9 Å2 (FIG. 4A and Table 8). Two Arg-Asp interactions govern the specificity of the interaction (FIG. 4A). This includes Arg91 in IL-9, which is also involved in the interaction with hIL-9Ra.

Next, two additional Fab:hIL-9 complexes were structurally determined (FIG. 4B and 4C and Table 7). Fab 6E2 binds hIL-9 with an average interface area of 987.6 Å2. The cytokine is placed with its A-helix in-between the light and heavy chain of the Fab (FIG. 4B and Table 8). The light chain of Fab 6E2 interacts specifically with the C-helix by coordinating Arg91 of the cytokine through Asp31 and Asp49 (FIG. 4B). Another Asp on the light chain, Asp95, interacts with the main chain of Leu24 placed at the tip of helix A. The heavy chain of Fab 6E2 adds to this interaction site by engaging with the A-helix and part of the D-helix. Further, Fab 7D6 mainly binds the A-helix, with the heavy chain covering the first half of the helix and the light chain the second half creating an interface area of 862.8 Å2 (FIG. 4C and Table 8). Even though the interaction mainly covers the A-helix, the light chain still engages Arg91 through an interaction with the main chain of Lys94 on the light chain (FIG. 4C).

Thus, all of the Fab:hIL-9 complexes engage Arg91 of the IL-9 in a specific interaction.

Next, to understand how the antibodies inhibit the binding of hIL-9 to its receptors and the different efficacies of the three antibodies in inhibiting the IL-9 signaling pathway, the structure of the binary hIL-9:hIL-9Ra complex with the structure of the Fab:hIL-9 complexes based on the superposition of hIL-9 in the respective structures was overlaid (FIG. 4D). All three Fabs, and therefore the corresponding full antibodies, show a partial overlap with the binding site of the receptor and in this way sterically hinder the binding of IL-9 to the receptor.

Antibody 7D6 performs poorly in inhibiting IL-9 in the cellular proliferation assay whereas 6E2 performs very well (IC50=2.1 nM versus 30.8 pM) (Table 6). Since the large difference in potency cannot just be simply explained by small difference in affinity (KD=1.8 nM for 7D6 versus 0.24 nM for 6E2), further analysis of the epitopes of these Fabs was performed. Both Fab fragments cover the A-helix extensively. However, their orientations on hIL-9 are perpendicular to each other. This results in Fab 6E2 holding hIL-9 in a crevice between its light and heavy chains, covering also the C-helix to a large extent (FIGS. 4B and 4D and Table 9). On the other hand, Fab 7D6 hardly covers the C-helix (FIG. 4C and 4D and Table 9). This availability of the C-helix might create an entry point for hIL-9Rα to engage hIL-9 through the C-helix and force the dissociation of the Fab from hIL-9.

Further analysis of the epitope of Fab 6D3 was also performed. This antibody than inhibited the IL-9 signal in the cellular reporter assay slightly less than antibody 6E2 (Table 6). The structure of Fab6D3:hIL-9 shows that Fab 6D3 binds mainly through the C-helix and leaves the A-helix almost completely available (FIGS. 4A and 4D). This indicates that availability of the A-helix is not enough to allow the receptor hIL-9Ra to efficiently bind hIL-9 and dislodge the Fab. Fab 6D3 does not completely cover the hIL-9Ra binding site on the C-helix, which could explain the difference in efficacy between mAb 6E2 and mAb 6D3 (FIG. 4D).

These results indicate that the efficacy of the antibodies in inhibiting the hIL-9:hIL-9Ra interaction and the hIL-9 signaling pathway, is correlated with the ability of the antibodies to cover the hIL-9Rα binding site on the C-helix of hIL-9. This shows that the C-helix is key in the interaction between hIL-9 and hIL-9Rα. Thus, efficient therapeutic neutralizing agents (e.g., anti-IL-9 antibodies) should target this C-helix since it is the main entry point for hIL-9Rα on hIL-9.

TABLE 7
Crystallographic data and refinement statistics

| Protein complex | Fab 6E2:hIL-9 | Fab 7D6:hIL-9 | Fab 6D3:hIL-9 | Fab 35D8:mIL-9 |
|---|---|---|---|---|
| Crystallization | 0.2M lithium sulfate, | 0.25M ammonium | 0.1M ammonium | 0.2M Zinc acetate, |
| conditions | 0.1M phosphate/citric | nitrate, 18% PEG | sulfate, 0.1M Tris, 16% | 23% PEG 3350 |
| | acid, pH 3.8 | 3350, 5% ethylene | PEG 1500, pH 5.8 | |
| | | glycol | | |
| Protein concentration | 13.4 mg/ml | 15 mg/ml | 14 mg/ml | 13.5 mg/ml |
| cryoprotectant | 20% glycerol | 20% ethylene glycol | 25% PEG 1500 | 20% DMSO/ethylene |
| | | | | glycol/glycerol (2:2:1) |
| Data Collection^{a} | | | | |
| X-ray source | ESRF (ID23-1) | ESRF (ID30A-3) | PETRA III (P14) | PETRA III (P13) |
| Wavelength (λ) | 0.97242 | 0.96770 | 0.9763 | 0.9762 |
| Space group | P1 | P2₁ | P2₁2₁2 | C222₁ |
| Cell dimensions | | | | |
| a, b, c (Å) | 69.37, 94.37, 107.34 | 81.35, 102.75, 164.37 | 60.48, 81.87, 116.55 | 70.21, 78.92, 192.10 |
| α, β, γ (°) | 82.53, 72.00, 80.79 | 90, 94.119, 90 | 90, 90, 90 | 90, 90, 90 |
| Resolution (Å) | 50-3.35 | 50-2.5 | 999-1.7 | 999-1.8 |
| | (3.55-3.35) | (2.65-2.5) | (1.79-1.7) | (1.9-1.8) |
| Rₘₑₐₛ (%) | 27.8 (112.1) | 11.4 (114.0) | 7.5 (162.6) | 13.1 (152.9) |
| <I/σ> | 4.78 (1.04) | 11.99 (1.23) | 21.59 (1.66) | 9.49 (1.06) |
| CC^{1/2} (%) | 96.0 (51.7) | 99.7 (49.5) | 99.9 (70.3) | 99.7 (44.3) |
| Completeness (%) | 93.6 (89.60) | 97.7 (95.9) | 99.2 (97.9) | 99.7 (99.1) |
| Redundancy | 2.9 (2.8) | 3.2 (3.2) | 13.4 (13.6) | 5.8 (5.8) |
| Wilson B (Å²) | 56.48 | 55.16 | 36.21 | 35.04 |
| Refinement^{b} | | | | |
| Resolution (Å) | 48.92-3.34 | 40.57-2.49 | 41.96-1.7 | 46.04-1.8 |
| No. reflections | 34587 | 92479 | 63802 | 49646 |
| No. reflections | 1728 | 4624 | 3188 | 2482 |
| used in Rfree | | | | |
| R_{work}/R_{free} (%) | 22.58/27.83 | 21.00/26.19 | 17.61/21.02 | 18.33/22.87 |
| No. non-H atoms | | | | |
| Protein | 16132 | 16289 | 4117 | 4087 |
| Ligand/ion | 25 | 0 | 25 | 23 |
| Water | 2 | 225 | 424 | 316 |
| R.m.s. deviations | | | | |
| Bond lengths (Å) | 0.005 | 0.011 | 0.010 | 0.008 |
| Bond angles (°) | 1.05 | 1.36 | 1.16 | 0.97 |
| Ramachandran | 95.31 | 94.61 | 98.32 | 97.94 |
| favored | | | | |
| Ramachandran | 4.31 | 4.82 | 1.68 | 1.68 |
| allowed | | | | |
| Ramachandran | 0.38 | 0.57 | 0.00 | 0.37 |
| outliers | | | | |
| Rotamer outliers | 0.00 | 0.00 | 0.00 | 0.00 |
| Clash score | 9.52 | 11.87 | 2.45 | 6.92 |
| B-factors (Å²) | | | | |
| Protein | 78.46 | 71.06 | 39.57 | 40.39 |
| Ligand/ion | 96.97 | 0 | 71.13 | 58.75 |
| Water | 25.75 | 52.14 | 45.12 | 42.41 |

TABLE 8
Overview of buried residues and hydrogen bonds at interaction interfaces.
Buried residues and hydrogen bonds of interaction interfaces as analyzed by PISA. For the
Fab:h/mIL-9 complexes, only the interfaces between Fab and h/mIL-9 were analyzed.

| hIL-9 | | hIL-9Rα | | | | | | | | | |
|---|---|---|---|---|---|---|---|---|---|---|---|
| N-term. | Gly20 | AB-loop | Asn60 | | | | | | | | |
| | Cys21 | | Ile61 | | | | | | | | |
| | Pro22 | | Leu62 | | | | | | | | |
| | Thr23 | CD-loop | Gln87 | | | | | | | | |
| αA | Leu24 | | Ala88 | | | | | | | | |
| | Ala25 | | Pro89 | | | | | | | | |
| | Leu28 | | Gly90 | | | | | | | | |
| | Asp29 | EF-loop | Leu106 | | | | | | | | |
| | Phe32 | | Pro108 | | | | | | | | |
| | Lys36 | | Glu109 | | | | | | | | |
| BC-loop | Arg84 | | Ala110 | | | | | | | | |
| | Tyr85 | | Val111 | | | | | | | | |
| αC | Pro86 | | Leu112 | | | | | | | | |
| | Leu87 | | Val113 | | | | | | | | |
| | Ile88 | | Pro114 | | | | | | | | |
| | Ser90 | | Ser115 | | | | | | | | |
| | Arg91 | | Asp116 | | | | | | | | |
| | Lys93 | | Phe118 | | | | | | | | |
| | Lys94 | BC2-loop | Pro170 | | | | | | | | |
| | Ser95 | | Ala171 | | | | | | | | |
| | Glu97 | | Leu172 | | | | | | | | |
| | Va198 | | Glu173 | | | | | | | | |
| | Asn101 | | Pro174 | | | | | | | | |
| CD-loop | Asn102 | | Met175 | | | | | | | | |
| αD | Lys136 | | Thr177 | | | | | | | | |
| | | | Leu178 | | | | | | | | |
| | | FG2-loop | Asp234 | | | | | | | | |
| | | | Val235 | | | | | | | | |
| | | | Val236 | | | | | | | | |
| | | | Glu238 | | | | | | | | |
| | | | Glu239 | | | | | | | | |
| | | | Tyr241 | | | | | | | | |
| Hydrogen bonds | | | | | | | | | | | |
| hIL-9 | | hIL-9Rα | | | | | | | | | |
| Arg91 | | Ala171 | | | | | | | | | |
| Leu24 | | Glu239 | | | | | | | | | |
| Lys94 | | Asp116 | | | | | | | | | |
| Lys94 | | Asp116 | | | | | | | | | |
| Lys94 | | Val111 | | | | | | | | | |
| Lys94 | | Val111 | | | | | | | | | |
| mIL-9 | | 35D8_{heavy} | | | | | | | | | |
| αA | Thr24 | CDR1 | Tyr33 | | | | | | | | |
| | Trp25 | | Asp35 | | | | | | | | |
| | Arg28 | CDR2 | Ala54 | | | | | | | | |
| | Asp29 | | Tyr55 | | | | | | | | |
| | Tyr32 | | Tyr60 | | | | | | | | |
| | Glu35 | CDR3 | His102 | | | | | | | | |
| | Asn36 | | Tyr103 | | | | | | | | |
| αC | Arg84 | | Ser104 | | | | | | | | |
| | Pro87 | | Asp105 | | | | | | | | |
| | Va188 | | Thr 106 | | | | | | | | |
| | His90 | | His107 | | | | | | | | |
| | Arg91 | | Gly108 | | | | | | | | |
| mIL-9 | | 35D8_{light} | | | | | | | | | |
| N-term. | Cys21 | | | | | | | | | | |
| | Ser22 | CDR1 | Gly28 | | | | | | | | |
| | Thr23 | | Asn29 | | | | | | | | |
| αA | Trp25 | | Phe30 | | | | | | | | |
| | Gly26 | | Lys65 | | | | | | | | |
| αC | Arg91 | CDR3 | Phe90 | | | | | | | | |
| | Arg94 | | Asp91 | | | | | | | | |
| | Ile95 | | Tyr92 | | | | | | | | |
| | Val98 | | Ile93 | | | | | | | | |
| | Leu99 | | Gly94 | | | | | | | | |
| | Ile102 | | | | | | | | | | |
| Hydrogen bonds | | | | | | | | | | | |
| mIL-9 | | 35D8_{heavy} | | | | | | | | | |
| Asn36 | | Tyr33 | | | | | | | | | |
| Arg28 | | Asp35 | | | | | | | | | |
| Arg28 | | Asp35 | | | | | | | | | |
| Arg91 | | His102 | | | | | | | | | |
| Arg91 | | His102 | | | | | | | | | |
| Tyr32 | | Tyr103 | | | | | | | | | |
| Arg91 | | Asp105 | | | | | | | | | |
| Arg91 | | Thr106 | | | | | | | | | |
| Asp29 | | Tyr103 | | | | | | | | | |
| Pro87 | | Thr106 | | | | | | | | | |
| mIL-9 | | 35D8_{light} | | | | | | | | | |
| Arg94 | | Gly28 | | | | | | | | | |
| hIL-9 | | 6D3_{heavy} | | hIL-9 | | 6E2_{heavy} | | hIL-9 | | 7D6_{heavy} | |
| αA | Leu 24 | | Arg32 | N-term. | Thr23 | CDR1 | Thr28 | N-term. | Cys21 | CDR1 | Thr30 |
| | Ala25 | CDR1 | Ala35 | αA | Leu24 | | Ser31 | | Pro22 | | Tyr33 |
| | Leu 28 | | Leu52 | | Ala25 | | Ser32 | | Thr23 | CDR2 | Phe52 |
| | Asp29 | CDR2 | Asp54 | | Ile27 | | Ala33 | αA | Leu24 | | Ala54 |
| | Phe32 | | Tyr55 | | Leu28 | | Trp47 | | Ala25 | | Arg55 |
| BC- | Arg84 | | Asp56 | | Asp29 | | Thr50 | | Ile27 | | Asp56 |
| loop | Tyr85 | | Ser58 | | Asn31 | CDR2 | Val52 | | Leu28 | | Ser58 |
| αC | Pro86 | | Val59 | | Phe32 | | Ser54 | | Asn31 | | Thr59 |
| | Leu87 | | Tyr60 | | Ile34 | | Ile55 | | Phe32 | | Ser60 |
| | Ile88 | CDR3 | Ala102 | | Asn35 | | Thr56 | | Asn35 | | Pro63 |
| | Ser90 | | Ala103 | | Gln38 | | Thr57 | αC | Arg91 | CDR3 | Trp106 |
| | Arg91 | | Thr104 | αC | Arg91 | | Thr58 | αD | Gln133 | | Tyr107 |
| | Lys94 | | Leu105 | αD | Leu129 | | Tyr59 | | Lys136 | | Tyr110 |
| | | | | | Glu130 | CDR3 | Leu99 | C-term. | Met140 | | Tyr111 |
| | | | | | Gln133 | | Ala100 | | Lys143 | | |
| | | | | | Lys136 | | Gly101 | | | | |
| | | | | | Met137 | | Pro102 | | | | |
| | | | | | | | Tyr103 | | | | |
| hIL-9 | | 6D3_{light} | | hIL-9 | | 6E2_{light} | | hIL-9 | | 7D6_{light} | |
| BC- | Asn78 | CDR1 | Ser27 | N-term. | Gly20 | CDR1 | Gly28 | αA | Ala25 | CDR1 | Leu24 |
| loop | Gln82 | | Gly29 | | Cys21 | | Ser29 | | Leu28 | | Ser26 |
| | Thr83 | | Ser30 | | Pro22 | | Tyr30 | | Asp29 | | Ser28 |
| | Arg84 | | Ser31 | | Thr23 | | Asp31 | | Phe32 | | Thr30 |
| | Tyr85 | | Tyr32 | αA | Leu24 | | His33 | | Asn35 | | Ser31 |
| αC | Pro86 | CDR2 | Asp51 | | Ala25 | CDR2 | Asp49 | | Lys36 | | Ser32 |
| | Leu87 | CDR3 | Tyr91 | | Leu28 | | Asn50 | AB- | Glu39 | | Asn33 |
| | Phe89 | | Asp92 | | Asp29 | | Arg65 | loop | | | |
| | Ser90 | | Ser93 | αC | Leu87 | CDR3 | Tyr90 | αC | Tyr85 | | Tyr34 |
| | | | Ser94 | | Ile88 | | Ser92 | | Ile88 | CDR3 | Lys94 |
| | | | Ala95 | | Arg91 | | Thr93 | | Arg91 | | Gly95 |
| | | | | | Lys94 | | Asp95 | | | | Ser96 |
| | | | | | Ser95 | | Ala96 | | | | Thr97 |
| | | | | | Va198 | | Leu97 | | | | |
| | | | | αD | Lys136 | | | | | | |

Hydrogen bonds

| hIL-9 | 6D3_{heavy} | hIL-9 | 6E2_{heavy} | hIL-9 | 7D6_{heavy} |
|---|---|---|---|---|---|
| Arg91 | Arg32 | Asn35 | Ser31 | Thr23 | Asp56 |
| Arg91 | Arg32 | hIL-9 | 6E2_{light} | hIL-9 | 7D6_{light} |
| Arg91 | Asp54 | Thr23 | Asp95 | Arg91 | Lys94 |
| Arg91 | Asp54 | Thr23 | Asp95 | Asp29 | Ser96 |
| Ser90 | Tyr60 | Leu24 | Asp95 | | |
| Ala25 | Arg32 | Arg91 | Asp31 | | |
| Asp29 | Arg32 | Arg91 | Asp49 | | |
| Asp29 | Arg32 | Arg91 | Asp49 | | |
| hIL-9 | 6D3_{light} | | | | |
| Thr83 | Ser30 | | | | |
| Arg84 | Gly29 | | | | |
| Tyr85 | Tyr32 | | | | |
| Arg84 | Tyr91 | | | | |

TABLE 9
Antibody heavy and light chain interaction with buried IL-9 Residues.

| | | 6D3 | | 6E2 | | 7D6 | | 35D8 | | | |
|---|---|---|---|---|---|---|---|---|---|---|---|
| hIL-9 C helix residues | | heavy | light | heavy | light | heavy | light | heavy | light | mIL-9 C helix residues | |
| 84 | R | X | X | | | | | X | | 84 | R |
| 85 | Y | X | X | | | | | | | 85 | L |
| 86 | P | X | X | | | | | | | 86 | L |
| 87 | L | X | X | | X | | | X | | 87 | P |
| 88 | I | X | | | X | | X | X | | 88 | V |
| 89 | F | | X | | | | | | | 89 | F |
| 90 | S | X | X | | | | | X | | 90 | H |
| 91 | R | X | | X | X | X | X | X | X | 91 | R |
| 92 | V | | | | | | | | | 92 | V |
| 93 | K | | | | | | | | | 93 | K |
| 94 | K | X | | | X | | | | X | 94 | R |
| 95 | S | | | | X | | | | X | 95 | I |
| 96 | V | | | | | | | | | 96 | V |
| 97 | E | | | | | | | | | 97 | F |
| 98 | V | | | | X | | | | X | 98 | V |
| 99 | L | | | | | | | | X | 99 | I |
| 100 | K | | | | | | | | | 100 | K |
| 101 | N | | | | | | | | | 101 | N |
| 102 | N | | | | | | | | | 102 | I |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed:

1. An isolated antibody that specifically binds to human or mouse IL-9, the antibody comprising:
(a) a VH comprising the CDRH1, CDRH2, and CDRH3 amino acid sequences of the VH amino acid sequence of SEQ ID NO: 42, 43, 44, 45, 46, 47, 48, 49, or 128; and/or
(b) a VL comprising the CDRL1, CDRL2, and CDRL3 amino acid sequences of the VL amino acid sequence of SEQ ID NO: 50, 51, 52, 53, 54, 55, 56, or 129.

2. The isolated antibody of claim 1, wherein the antibody comprises the CDRH1, CDRH2, CDRH3, CDRL1, CDRL2 and CDRL3 amino acid sequences of the VH and VL amino acid sequences of SEQ ID NOs: 42 and 51; 43 and 55; 44 and 54; 45 and 53; 46 and 50; 47 and 52;
48 and 50; 49 and 56; or 128 and 129, respectively.

3. The isolated antibody of claim 1 or 2, wherein CDRH1, CDRH2 and CDRH3 comprise the CDRH1, CDRH2 and CDRH3 amino acid sequences, respectively, set forth in SEQ ID NOs: 1, 2, and 3; 4, 5, and 6; 7, 8, and 9; 10, 11, and 12; 13, 14, and 15; 16, 17, and 18; 19, 20, and 21; or 122, 123, and 124.

4. The isolated antibody of any one of claims 1-3, wherein CDRL1, CDRL2 and CDRL3 comprise the CDRL1, CDRL2 and CDRL3 amino acid sequences, respectively, set forth in SEQ ID NOs: 22, 23, and 24; 25, 26, and 27; 28, 29, and 30; 31, 29, and 32; 33, 34, and 35; 36, 37, and 38; 39, 40, and 41; or 125, 126, and 127.

5. The isolated antibody of any one of claims 1-4, wherein the antibody comprises the CDRH1, CDRH2, CDRH3, CDRL1, CDRL2, and CDRL3 amino acid sequences, respectively, set forth in SEQ ID NOs: 1, 2, 3, 22, 23, and 24; 4, 5, 6, 25, 26, and 27; 7, 8, 9, 28, 29, and 30; 7, 8, 9, 31, 29, and 32; 10, 11, 12, 33, 34, and 35; 13, 14, 15, 36, 37, and 38; 16, 17, 18, 39, 40, and 41; 19, 20, 21, 36, 37, and 38; or 122, 123, 124, 125, 126, and 127.

6. The isolated antibody of any one of claims 1-5, wherein the antibody comprises the VH amino acid sequence of SEQ ID NO: 42, 43, 44, 45, 46, 47, 48, 49, or 128.

7. The isolated antibody of claim 6, wherein the amino acid sequence of the VH consists of the amino acid sequence of SEQ ID NO: 42, 43, 44, 45, 46, 47, 48, 49, or 128.

8. The isolated antibody of any one of claims 1-5, wherein the antibody comprises the VL amino acid sequence of SEQ ID NO: 50, 51, 52, 53, 54, 55, 56, or 129.

9. The isolated antibody of claim 8, wherein the amino acid sequence of the VL consists of the amino acid sequence of SEQ ID NO: 50, 51, 52, 53, 54, 55, 56, or 129.

10. An isolated antibody that specifically binds to human or mouse IL-9, the antibody comprising a VH comprising the amino acid sequence of SEQ ID NO: 42, 43, 44, 45, 46, 47, 48, 49, or 128, and a VL comprising the amino acid sequences of SEQ ID NO 50, 51, 52, 53, 54, 55, 56, or 129.

11. The isolated antibody of claim 10, wherein the amino acid sequence of the VH consists of the amino acid sequence of SEQ ID NO: 42, 43, 44, 45, 46, 47, 48, 49, or 128, and the amino acid sequence of the VL consists of the amino acid sequence of SEQ ID NO: 50, 51, 52, 53, 54, 55, 56, or 129.

12. The isolated antibody of claim 10 or claim 11, wherein the antibody comprises the VH and VL amino acid sequences of SEQ ID NOs: 42 and 51; 43 and 55; 44 and 54; 45 and 53; 46 and 50; 47 and 52; 48 and 50; 49 and 56; or 128 and 129, respectively.

13. The isolated antibody of claim 12, wherein the amino acid sequences of the VH and VL consists of the amino acid sequence of SEQ ID NOs: 42 and 51; 43 and 55; 44 and 54; 45 and 53; 46 and 50; 47 and 52; 48 and 50; 49 and 56; or 128 and 129, respectively.

14. The isolated antibody of any one of claims 1-13, wherein the antibody comprises a heavy chain constant region selected from the group consisting of human IgG₁, IgG₂, IgG₃, IgG₄, IgA₁, and IgA₂.

15. The isolated antibody of any one of the preceding claims, wherein the antibody comprises a heavy chain constant region that is a variant of a wild-type heavy chain constant region, wherein the variant heavy chain constant region binds to an FcγR with higher affinity than the wild-type heavy chain constant region binds to the FcγR.

16. The isolated antibody of claim 15, wherein the FcγR is FcγRIIB or FcγRIIIA.

17. The isolated antibody of claim 14, wherein the amino acid at position 297 of the heavy chain constant region, according to the EU numbering system, is A or Q.

18. The isolated antibody of claim 14, wherein the amino acids at positions 234 and 235 of the heavy chain constant region, according to the EU numbering system, are both A.

19. The isolated antibody of claim 14, wherein the amino acids at positions 433, 434, and 436 of the heavy chain constant region, according to the EU numbering system, are K, F, and Y, respectively.

20. The isolated antibody of claim 14, wherein the amino acids at positions 252, 254, and 256 of the heavy chain constant region, according to the EU numbering system, are Y, T, and E, respectively.

21. The isolated antibody of claim 14, wherein the amino acids at positions 428 and 434 of the heavy chain constant region, according to the EU numbering system, are L and S, respectively.

22. The isolated antibody of claim 14, wherein the amino acid at positions 309, 311, and 434 of the heavy chain constant region, according to the EU numbering system, are D, H, and S, respectively.

23. The isolated antibody of any one of claims 1-22, wherein the antibody inhibits binding of human IL-9 to human IL-9Rα.

24. The isolated antibody of any one of claims 1-23, wherein the antibody binds to human IL-9 with a K_{D} of less than 1 nM.

25. The isolated antibody of any one of the preceding claims, wherein the antibody is bispecific.

26. The isolated antibody of any one of the preceding claims, wherein the isolated antibody is conjugated to a cytotoxic agent, cytostatic agent, toxin, radionuclide, or detectable label.

27. An isolated polynucleotide encoding the VH and/or the VL, or a heavy chain and/or a light chain, of the isolated antibody of any one of the preceding claims.

28. A vector comprising the polynucleotide of claim 27.

29. A recombinant host cell comprising:
(a) the polynucleotide of claim 27;
(b) the vector of claim 28;
(c) a polynucleotide encoding the VH and the VL, or a heavy chain and a light chain, of the isolated antibody of any one of claims 1-26;
(d) a vector comprising a polynucleotide encoding the VH and the VL, or a heavy chain and a light chain, of the isolated antibody of any one of claims 1-26;
(e) a first polynucleotide encoding the VH or a heavy chain of the isolated antibody of any one of claims 1-26, and a second polynucleotide encoding the VL or a light chain of isolated antibody of any one of claims 1-26; or
(f) a first vector comprising a first polynucleotide encoding the VH or a heavy chain of the isolated antibody of any one of claims 1-26, and a second vector comprising a second polynucleotide encoding the VL or a light chain of the isolated antibody of any one of claims 1-26.

30. A pharmaceutical composition comprising the isolated antibody of any one of claims 1-26, the polynucleotide of claim 27, the vector of claim 28, or the host cell of claim 29, and a pharmaceutically acceptable carrier or excipient.

31. A method of producing an isolated antibody, the method comprising culturing the host cell of claim 30 under suitable conditions so that the polynucleotide is expressed, and the isolated antibody is produced.

32. A method of producing an isolated antibody, the method comprising expressing in a cell:
(a) a first polynucleotide encoding the VH of the antibody of any one of claims 1-26 and a second polynucleotide encoding the VL of the antibody of any one of claims 1-26; or
(b) a first polynucleotide encoding a heavy chain of the antibody of any one of claims 1-26 and a second polynucleotide encoding a light chain of the antibody of any one of claims 1-26,
under suitable conditions so that the polynucleotides are expressed, and the antibody is produced.

33. A method of antagonizing the interaction of human or mouse IL-9 with an IL-9 receptor in a subject, the method comprising administering to the subject an effective amount of the isolated antibody of any one of claims 1-26, the polynucleotide of claim 27, the vector of claim 28, the host cell of claim 29, or the pharmaceutical composition of claim 30.

34. A method of treating an inflammatory disease in a subject, the method comprising administering to the subject an effective amount of the isolated antibody of any one of claims 1-26, the polynucleotide of claim 27, the vector of claim 28, the host cell of claim 29, or the pharmaceutical composition of claim 30.

35. A method of treating cancer in a subject, the method comprising administering to the subject an effective amount of the isolated antibody of any one of claims 1-26, the polynucleotide of claim 27, the vector of claim 28, the host cell of claim 29, or the pharmaceutical composition of claim 30.

36. A method of treating an autoimmune disease in a subject, the method comprising administering to the subject an effective amount of the isolated antibody of any one of claims 1-26, the polynucleotide of claim 27, the vector of claim 28, the host cell of claim 29, or the pharmaceutical composition of claim 30.

37. The method of any one of claims 33-36, wherein the isolated antibody, polynucleotide, vector, host cell, or pharmaceutical composition is administered, systemically, intravenously, subcutaneously, intratumorally, or is delivered to a tumor draining lymph node.

38. The method of any one of claims 33-36, further comprising administering an additional therapeutic agent to the subject.

39. An isolated antibody according to any one of claims 1-26, a polynucleotide according to claim 27, a vector according to claim 28, a host cell according to claim 29, or a pharmaceutical composition according to claim 30 for use in a method according to any one of claims 33-38.

40. Use of an isolated antibody according to any one of claims 1-26, a polynucleotide according to claim 27, a vector according to claim 28, a host cell according to claim 29 in the manufacture of a medicament for the treatment of inflammatory disease, cancer or autoimmune disease in a subject.
